SEQUENCE LISTING (17 sequences)

SEQ ID NO: 1 - protein, 4 aa, Artificial Sequence
  A synthetic peptide
LLVY

SEQ ID NO: 2 - protein, 4 aa, Artificial Sequence
  A synthetic peptide
  SITE 1, 3: Xaa = norleucine
XPXD

SEQ ID NO: 3 - protein, 8 aa, Artificial Sequence
  A synthetic peptide
DEVDDEVD

SEQ ID NO: 4 - protein, 5 aa, Artificial Sequence
  A synthetic peptide
  SITE 1: Xaa = Trp or Leu
  SITE 5: Xaa = any amino acid
XEHDX

SEQ ID NO: 5 - protein, 5 aa, Artificial Sequence
  A synthetic peptide
  SITE 3, 5: Xaa = any amino acid
DEXDX

SEQ ID NO: 6 - protein, 5 aa, Artificial Sequence
  A synthetic peptide
  SITE 1: Xaa = Leu or Val
  SITE 3, 6: Xaa = any amino acid
XEXDX

SEQ ID NO: 7 - protein, 5 aa, Artificial Sequence
  A synthetic peptide
  SITE 5: Xaa = any amino acid
IEGRX

SEQ ID NO: 8 - protein, 7 aa, Artificial Sequence
  A synthetic peptide
  SITE 3, 5: Xaa = any amino acid
  SITE 7: Xaa = Ser or Gly
ENXYXQX

SEQ ID NO: 9 - protein, 5 aa, Artificial Sequence
  A synthetic peptide
  SITE 5: Xaa = any amino acid
PRNKX

SEQ ID NO: 10 - protein, 14 aa, Artificial Sequence
  A synthetic peptide
  SITE 6: Xaa = Lys or Asn
  SITE 7: Xaa = Met or Leu
EISEVXXDAEFRHD

SEQ ID NO: 11 - protein, 10 aa, Artificial Sequence
  A synthetic peptide
SEVNLDAEFR

SEQ ID NO: 12 - protein, 6 aa, Artificial Sequence
  A synthetic peptide
AAFAAF

SEQ ID NO: 13 - protein, 4 aa, Artificial Sequence
  A synthetic peptide
GFGF

SEQ ID NO: 14 - protein, 4 aa, Artificial Sequence
  A synthetic peptide
EAAF

SEQ ID NO: 15 - protein, 5 aa, Artificial Sequence
  A synthetic peptide
RLRGG

SEQ ID NO: 16 - protein, 4 aa, Artificial Sequence
  A synthetic peptide
LRGG

SEQ ID NO: 17 - protein, 8 aa, Artificial Sequence
  A synthetic peptide
RPFHLLVY

(19) United States
(12) Patent Application Publication
(10) Pub. No.: US 2007/0048812 A1
(43) Pub. Date: Mar. 1, 2007

(54) Cell-based luminogenic and nonluminogenic proteasome assays

(76) Inventors: Richard A. Moravec, Oregon, WI (US); Terry L. Riss, Oregon, WI (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/510,278

(22) Filed: Aug. 25, 2006

Related U.S. Application Data
(60) Provisional application No. 60/713,906, filed on Sep. 1, 2005.

(51) Int. Cl. C12Q 1/66

(52) U.S. Cl. 435/8

(57) ABSTRACT

A method to detect proteasome activity in permeabilized cells, and optionally in a multiplex assay to detect presence or amount of at least one molecule for a different enzyme-mediated reaction, is provided.

### CROSS-REFERENCE TO RELATED APPLICATIONS

This application claims benefit under 35 U.S.C. 119(e) of U.S. Provisional Application Ser. No. 60/713,906, filed Sep. 1, 2005, which is incorporated herein by reference in its entirety.

### BACKGROUND

Luminescence is produced in certain organisms as a result of a luciferase-mediated oxidation reaction. Luciferase genes from a wide variety of vastly different species, particularly the luciferase genes of Photinus pyralis and Photuris pennsylvanica (fireflies of North America), Pyrophorus plagiophthalamus (the Jamaican click beetle), Renilla reniformis (the sea pansy), and several bacteria (e.g., Xenorhabdus luminescens and Vibrio spp), are extremely popular luminescence reporter genes. Firefly luciferase is also a popular reporter for determining ATP concentrations, and, in that role, is widely used to detect biomass. Luminescence is also produced by other enzymes when those enzymes are mixed with certain synthetic substrates, for instance, alkaline phosphatase and adamantyl dioxetane phosphate, or horseradish peroxidase and luminol.

Luciferase genes are widely used as genetic reporters due to the non-radioactive nature, sensitivity, and extreme linear range of luminescence assays. For instance, as few as 10⁻²⁰ moles of firefly luciferase can be detected. Consequently, luciferase assays of gene activity are used in virtually every experimental biological system, including both prokaryotic and eukaryotic cell cultures, transgenic plants and animals, and cell-free expression systems. Similarly, luciferase assays used to determine ATP concentration are highly sensitive, enabling detection to below 10⁻¹⁶ moles.

Luciferases can generate light via the oxidation of enzyme-specific substrates, e.g., luciferins. For firefly luciferase and all other beetle luciferases, light generation occurs in the presence of magnesium ions, oxygen, and ATP. For anthozoan luciferases, including Renilla luciferase, only oxygen is required along with the substrate coelentrazine. Generally, in luminescence assays to determine genetic activity, reaction substrates and other luminescence activating reagents are introduced into a biological system suspected of expressing a reporter enzyme. Resultant luminescence, if any, is then measured using a luminometer or any suitable radiant energy-measuring device. The assay is very rapid and sensitive, and provides gene expression data quickly and easily, without the need for radioactive reagents.

Reporters are also useful to detect the presence or activity of molecules within cells or supernatants. For instance, proteases constitute a large and important group of enzymes involved in diverse physiological processes such as protein turnover in blood coagulation, inflammation, reproduction, fibrinolysis, and the immune response. Numerous disease states are caused by, and can be characterized by, the alterations in the activity of specific proteases and their inhibitors. The ability to measure these proteases in research or in a clinical setting is significant to the investigation, treatment and management of disease states. For example, caspase-3 and caspase-7 are members of the cysteine aspartyl-specific protease (also known as the aspartate specific-cysteine protease, “ASCP”) family and play key effector roles in cell death in mammalian cells (Thornberry et al., 1992; Nicholson et al., 1995; Tewari et al., 1995; and Fernandes-Alnemri et al., 1996).

Proteases, however, are not easy to assay with their naturally occurring substrates. Moreover, many currently available synthetic substrates are expensive, insensitive, and nonselective.

Numerous chromogenic and fluorogenic substrates have been used to measure proteases (Monsees et al., 1994; Monsees et al., 1995) and modified luciferins have provided alternatives to fluorescent indicators (U.S. Pat. Nos. 5,035,999 and 5,098,828). Methods for using modified luciferins with a recognition site for a hydrolase as a pro-substrate were first described by Miska and Geiger (1989), where heterogeneous assays were conducted by incubating a modified luciferin with a hydrolase for a specified period of time, then transferring an aliquot of the mixture to a solution containing luciferase. Masuda-Nishimura et al. (2000) reported the use of a single tube (homogeneous) assay which employed a β-galactosidase substrate-modified luciferin.

Proteasomes are large multi-subunit enzyme complexes (sometimes called proteolytic machines) that perform the proteolytic function of the multicatalytic ubiquitin-proteasome pathway in the cytoplasm and nucleus of eukaryotic cells. The 20 S proteasome is part of a larger 26 S proteasome complex that forms a hollow cylinder composed of 4 stacked rings. 2 inner β-rings each contain 3 different proteolytic sites; 2 chymotrypsin-like sites cleave peptide bonds after hydrophobic residues, 2 trypsin-like sites cleave after basic residues, and 2 caspase-like (acidic/peptidylglutamyl peptide hydrolase) sites cleave after acidic residues. Proteasomes are found both in the cytosol and the nucleus.

The multicatalytic ubiquitin-proteasome pathway tightly regulates the turnover of proteins involved in normal cell cycling and signal transduction events such as the degradation of I-κB necessary to activate NF-κB. The pathway also is involved with antigen processing and presentation. Proteasome activity is critical for normal cell survival and function. Inhibition of the proteasome pathway results in apoptotic cell death. Because the ubiquitin-proteasome pathway is vital for cell cycling, function, and survival, it has become recognized as a therapeutic target in cancer, as malignant, transformed, and proliferating cells are more susceptible to proteasome inhibition than normal cells. Several proteasome inhibitors have been identified that are more toxic for tumor cells compared to normal cell counterparts. Moreover, one of those inhibitors, bortezomib (also known as PS-341 or Velcade), has received FDA approval for treatment of advanced multiple myeloma. This success has confirmed the proteasome as a valid target for development of cancer therapeutics.

Current proteasome assays, e.g., to screen for inhibitors, involve the use of preparations that have been purified from blood or frozen cell preparations and so may not correlate to activity in intact cells. Thus, there is a need for an improved proteasome assay. cl SUMMARY OF THE INVENTION

The invention provides for detection of one or more proteolytic activities associated with proteasomes in a cell-based luminogenic or nonluminogenic, e.g., fluorescent, assay. The invention also provides for multiplexing of nonluminogenic and luminogenic assays, e.g., in the same well, at least one of which is a cell-based assay for detection of one or more proteolytic proteasome activities. For example, a cell-based proteasome assay may be multiplexed with assays to detect the amount (e.g., activity) or presence of one or more moieties (molecules or activities) other than proteasomes expressed by a cell, including cofactors for enzymatic reactions such as ATP, proteins (peptides or polypeptides) that bind to and/or alter the conformation of a molecule, e.g., proteins that modify or cleave a peptide or polypeptide substrate, or a molecule which is bound by and/or altered by a protein, e.g., a substrate. The other assays may be cell-based or may be conducted after cell lysis. Moreover, the assays described herein may be employed with other assays, including reporter assays, nucleic-acid based assays or immunological-based assays. Further provided is a cell-based nonluminogenic or luminogenic assay to detect one or more cytosolic enzymes (intracellular enzymes not present in membrane bound cellular organelles or compartments) which employs a cell membrane permeabilization reagent in an amount which does not substantially disrupt intracellular membrane bound organelles or compartments in cells.

Thus, the invention provides a method to detect a proteolytic activity associated with proteasomes in a cell. A sample employed in the methods of the invention includes a eukaryotic cellular sample which is permeabilized, including a sample obtained from in vitro cultured cells or a physiological sample. In one embodiment, the method includes providing a reaction mixture for a beetle luciferase-mediated reaction which comprises eukaryotic cells, a cell membrane permeabilization reagent in an amount which does not substantially disrupt intracellular membrane bound organelles or compartments in the cells, and a luminogenic substrate for a proteasome associated protease. Proteolysis of the luminogenic substrate by the protease yields a luminogenic product that is a substrate for a beetle luciferase. Luminescence in the mixture is then detected or determined. The invention also provides a method in which a sample comprising intact eukaryotic cells and a cell membrane permeabilization reagent in an amount which does not substantially disrupt intracellular membrane bound organelles or compartments in the cells, is contacted with a reaction mixture for a beetle luciferase-mediated reaction which comprises a luminogenic substrate for a proteasome associated protease. Proteolysis of the luminogenic substrate by the protease yields a product that is a substrate for a beetle luciferase. Then luminescence in the mixture is detected or determined.

For instance, in one embodiment, a beetle luciferase and an appropriate luciferin, aminoluciferin, or a derivative thereof which is modified to contain a protease recognition site (modified, for example, via a covalent bond) for one of the protease activities associated with proteasomes, may be employed in a luminogenic assay to detect the activity of proteasomes in a cell. In one embodiment, the luminogenic assay reagent may be LLVY-aminoluciferin (LLVY; SEQ ID NO:1), LRR-aminoluciferin, nLPnLD-aminoluciferin (nLPnLD; SEQ ID NO:2), or may be another luminogenic proteasome associated protease substrate, e.g., a different peptide or polypeptide substrate linked to luciferin, aminoluciferin or a derivative thereof. Luciferin derivatives within the scope of the invention include, but are not limited to, derivatives which are substrates for a beetle luciferase, such as those described in U.S. application Ser. Nos. 60/685,957, 60/693,034, and 60/692,925, and U.S. published application 20040171099, the disclosures of which are incorporated by reference herein.

In another embodiment, the invention provides a reaction mixture comprising eukaryotic cells, a cell membrane permeabilization reagent in an amount which does not substantially disrupt intracellular membrane bound organelles or compartments in the cells, and a fluorogenic substrate for a proteasome associated protease. Fluorescence in the mixture is then detected or determined. For instance, a substrate for a proteasome associated protease may be modified to contain a fluorophore that emits light of a certain wavelength only after the enzyme reacts with the substrate and the fluorophore is contacted with (exposed to) light of a certain wavelength or range of wavelengths, e.g., LLVY-AMC (SEQ ID NO:1) is a fluorogenic substrate useful to detect the chymotrypsin activity of proteasomes, and cleavage of that substrate by a proteasome associated protease may be monitored via fluorescence of AMC.

The invention thus provides a single addition, homogeneous assay that can monitor proteasome activity in permeabilized eukaryotic cells. The presence of other cellular components such as those in close proximity to proteasomes in permeabilized cells, such as digitonin permeabilized cells, may provide an advantage over measuring proteasome activity in cell extracts or purified proteasomes. Moreover, permeabilized cells may be useful to measure other molecules or activities such as cytoplasmic enzymatic activities, e.g., selected P450 activities. The assay of the invention may be formatted such that luminescence or fluorescence is used to measure proteasome activity alone or in combination with measuring or detecting another molecule, e.g., measuring or detecting caspase activity or nucleic acid, by a luminescent/fluorescent multiplex assay or another multiplex assay. Thus, measures of cell viability (CellTiter-Glo™, CellTiter-Blue™, or CytoTox-ONE™) or specific measures of apoptotic cytotoxicity (Caspase-Glo™ 3/7, -8, -9 or Apo-ONE™) may be multiplexed with the proteasome assay. To detect those other molecules, the permeabilized cells may be subjected to conditions which lyse those cells and that lysate may be assayed for the one or more molecules, or subjected to a fractionation and/or purification of one or more fractions, which fraction may be assayed for the one or more molecules. In another embodiment, prior to permeabilization, the cellular sample may be assayed for a cell surface molecule and/or a molecule that is not bound to the cell surface, e.g., molecules in the supernatant. The molecules to be detected or employed in detection in single or multiplex assays may be native molecules or recombinant molecules, e.g., including fusion proteins. For instance, for luciferase-mediated reactions, the luciferase may be native or recombinant.

Therefore, in one embodiment, a combined luminogenic/nonluminogenic assay format of the present invention allows multiplexing of assays for one or more peptides or polypeptides, e.g., enzymes, one or more molecules which are bound by and/or altered by the peptide(s) or polypeptide(s), e.g., a peptide or polypeptide substrate for at least one enzyme, and/or one or more cofactors for an enzyme-mediated reaction, or other molecules or conditions, or a combination thereof. In one embodiment, the invention provides a method to detect the activity of a protease associated with proteasomes (a first enzyme-mediated reaction) and the presence or amount of a second molecule for a second enzyme-mediated reaction, and optionally other molecules including molecules for other enzyme-mediated reactions. The method includes contacting a cellular sample with a reaction mixture for the first enzyme-mediated reaction and the second enzyme-mediated reaction. In one embodiment, a cell membrane permeabilization reagent in an amount which does not substantially disrupt intracellular membrane bound organelles or compartments is added to the cells before the cells are contacted with the reaction mixture. In another embodiment, a cell membrane permeabilization reagent in an amount which does not substantially disrupt intracellular membrane bound organelles or compartments is added to the reaction mixture before the reaction mixture is contacted with the cells. In one embodiment, luminescence is employed to detect proteasome activity and fluorescence or colorimetry is employed to detect a molecule for a second enzyme-mediated reaction. The activity of proteasomes and the presence or amount of the second molecule are then detected.

Alternatively, a reaction mixture for the first reaction and for the second reaction may be added sequentially, where the first reaction mixture may include a cell membrane permeabilization reagent in an amount which does not substantially disrupt intracellular membrane bound organelles or compartments. Such a two-step assay may include reagent adjustment as specific buffer conditions can vary with the molecule(s) being detected. For example, reagent adjustment can include addition of a quenching agent for the first reaction, and/or an enhancing agent for the second reaction. In one embodiment, where at least two enzyme activities are detected, the two enzymes do not have the same activity, e.g., they do not bind to or react with the same substrate, or if they bind to and react with the same substrate, conditions or substrates are employed so that one of the enzymes does not react substantially with a substrate for the other enzyme, thereby providing for specificity. For example, a LLVY (SEQ ID NO:1) containing substrate may be a substrate for chymotrypsin or calpain. However, calpain-mediated reactions require calcium while chymotrypsin-mediated reactions do not. Thus, chymotrypsin activity may be detected with a LLVY (SEQ ID NO:1) containing substrate in an assay conducted under substantially calcium-free conditions.

The invention also provides for simultaneous or sequential detection of molecules or activities in a multiplex assay, including simultaneous or sequential detection of the activity, presence or amount of at least two molecules in concurrent reactions or sequential reactions, optionally without quenching one of the reactions or enhancing/accelerating one of the reactions. In one embodiment, a substrate for a proteasome associated protease and a reagent useful to detect another molecule, e.g., detect nucleic acid, are added to a cellular sample simultaneously and proteasome activity is detected before the amount or presence of the other molecule is detected. In another embodiment, substrates for two different enzyme-mediated reactions are added to a cellular sample simultaneously and proteasome activity is detected before the amount or presence of an enzyme, substrate and/or cofactor for a different enzyme-mediated reaction is detected. In yet another embodiment, a substrate for a proteasome associated protease and a reagent useful to detect another molecule are added to the sample simultaneously and the protease activity and the presence or amount of the other molecule detected at the same time. In another embodiment, substrates for two different enzyme-mediated reactions are added to the sample simultaneously and the presence or amount of an enzyme, substrate and/or cofactor for a different enzyme-mediated reaction, is detected at the same time that proteasome activity is detected. Alternatively, a substrate for a proteasome associated protease is added to a reaction mixture, the activity of that protease is detected, then a reagent useful to detect another molecule is added and the presence or amount of that molecule detected. In another embodiment, a substrate for a proteasome associated protease is added to a reaction mixture, the activity of that protease is detected, then a substrate for another enzyme-mediated reaction is added and the presence or amount of an enzyme, substrate and/or cofactor for that second reaction detected. Preferably, the activity, presence or amount of enzymes, substrates, cofactors and/or other molecules are detected in a reaction in a single receptacle, e.g., a well.

Thus, the invention provides multiplex assays where, in addition to detecting or determining one or more proteasome associated protease activities in a mixture which includes cell membrane permeabilized cells, the presence or amount of a co-factor, substrate or enzyme for another enzyme-mediated reaction may be detected or determined. In another embodiment, in addition to detecting or determining one or more proteasome associated protease activities in a mixture which includes cell membrane permeabilized cells, the presence or amount of a moiety associated with a nonenzymatic reaction may be detected or determined. In yet another embodiment, in addition to detecting or determining one or more proteasome associated protease activities in a mixture which includes cell membrane permeabilized cells, the presence or amount of a different cellular molecule, e.g., nucleic acid or protein, is detected or determined, for instance, by contacting the mixture with a reagent useful to detect that cellular molecule. Such a reagent includes a reagent that binds to the particular cellular molecule. Accordingly, in one embodiment, a nucleic acid binding dye may be employed to detect the presence or amount of nucleic acid in an assay for proteasome specific proteolytic activities.

Moreover, as certain cell permeabilization regents allow for differential solubilization, the invention provides for multiplex assays in which the cytosolic activity of at least two molecules may be detected or determined.

Also provided are compositions and kits which include one or more reagents for use in the assays of the invention. In one embodiment the composition is a solution, e.g., a solution in which one or more substrates are present at 0.005 to about 1.0 M, e.g., 0.05 to about 0.2 M.

The assay also has use as a drug discovery tool. Thus, the presence or amount of a modulator, for instance, an inhibitor, of a protease associated with proteasomes may be detected using an assay of the invention, e.g., a fluorogenic or luminogenic assay. The invention thus provides a method in which one or more agents are contacted with a reaction mixture comprising cells, a cell membrane permeabilization reagent in an amount which does not substantially disrupt intracellular membrane bound organelles or compartment in the cells, and a luminogenic or fluorogenic substrate for a protease associated with proteasomes, so as to yield a mixture. Also provided is a method in which one or more agents are contacted with cells, and that mixture contacted with a reaction mixture comprising a cell membrane permeabilization reagent in an amount which does not substantially disrupt intracellular membrane bound organelles or compartment in cells, and a luminogenic or fluorogenic substrate for a protease associated with proteasomes. Luminescence or fluorescence in the mixture is compared to luminescence or fluorescence in a corresponding mixture which lacks the one or more agents.

### BRIEF DESCRIPTION OF THE DRAWINGS

FIG. 1. Luminescent chymotrypsin-, trypsin-, and caspase-like proteasome activities in U266 cells following inhibitor treatment (AdaAhxL₃VS). Approximately 35,000 U266 cells were exposed to inhibitor for 1.5 hours and then contacted with 10 μM substrate.

FIGS. 2A-B. A) Lactacystin activity on proteasomes or calpain. After a 0.5 hour preincubation in 25 mM HEPES/0.5 mM EDTA/1 mM DTT, a 2×reagent was added (20 μM LLVY-aminoluciferin (SEQ ID NO:1)/3 mM CaCL₂/10 mM MgSO₄). B) Lactacystin or calpeptin activity on proteasomes. HL-60 cells were treated for 1.5 hours at 37° C. with lactacystin or calpeptin prior to addition of substrate.

FIG. 3. Luminescence versus proteasome inhibitor concentration in Jurkat cells. Jurkatt cells were exposed to inhibitor (AdaAhxL₃VS) for 1.5 hours and then contacted with LLVY-aminoluciferin (final concentration of 10 μM; SEQ ID NO:1).

FIGS. 4A-B. Comparison of luminescent and fluorescent sensitivities with 20 μM aminoluciferin-LLVY (SEQ ID NO:1) or 20 μM LLVY-AMC (SEQ ID NO:1), respectively, and HL-60 (A) and U937 (B) cells. Cells were permeabilized with 20 μg/ml digitonin. HL-60 cells were maintained at 22° C. Fluorescence data for U937 cells was from a 45 minute time point.

FIGS. 5A-C. A) Luminescence versus cell number. HL-60 cells maintained at 22° C. were permeabilized with 20 μg/ml digitonin and contacted with 20 μM LLVY-aminoluciferin (SEQ ID NO:1). B) Kinetics of luminescence with an aminoluciferin-LLVY (SEQ ID NO:1) substrate (20 μM) in U937 cells maintained at 22° C. and treated with 20 μg/ml digitonin. C) Kinetics of luminescence with an aminoluciferin-LLVY (SEQ ID NO:1) substrate (20 μM) in HL-60 cells maintained at 22° C. and treated with 20 μg/ml digitonin.

FIGS. 6A-B. Inhibition of luminescence in U937 (A) or HL-60 (B) cells by lactacystin. Approximately 50,000 U937 cells were treated with 20 μM LLVY-aminoluciferin (SEQ ID NO:1) and 20 μg/ml digitonin. Approximately 25,000 HL-60 cells in medium containing 10% FBS were treated with substrate and 20 μg/ml digitonin.

FIGS. 7A-B. Digitonin titration in medium with 5% FBS (A) or 10% FBS (B). Approximately 25,000 lactacystin treated HL-60 cells in medium containing 5% or 10% FBS were contacted with substrate and various amounts of digitonin. Data is from a 15 minute time point.

FIGS. 8A-B. Proteasome chymotrypsin activity and ATP content (viability) in HL-60 cells (A) or U937 cells (B) in the presence of various concentrations of lactacystin and in the presence (20 μg/ml) or absence of digitonin. Lactacystin treatment was for 1.5 hours and data is from a 16 or 18 minute time point for HL-60 and U937 cells, respectively. Cell viability measurements were made using CellTiter Glo (Promega Corp.) in a parallel series of samples to confirm that viability was not affected by lactacystin treatment.

FIGS. 9A-B. Multiplex assay measuring proteasome chymotrypsin-like activity and caspase 3/7 activity. (A) Luminescence data from approximately 50,000 Jurkat cells treated for 4.5 hours with lactacystin, and LLVY-aminoluciferin (SEQ ID NO:1) and (Z-DEVD₂)-R110 (10 μM final concentration for each substrate; (DEVD)₂; (SEQ ID NO:3)). (B) Fluorescence data from approximately 50,000 Jurkat cells treated for 4.5 hours with lactacystin, and LLVY-aminoluciferin (SEQ ID NO:1) and (Z-DEVD₂)-R110 (10 μM final concentration for each substrate; (SEQ ID NO:3)).

FIGS. 10A-C. Screen for cell membrane permeabilization reagents suitable to detect proteasomes in a cell-based luminescent reaction. Approximately 25,000 HL-60 cells in the absence of lactacystin treatment (panels A and B) or 25,000 U937 cells after lactacystin treatment (panel C) were incubated at 22° C. with a detergent and substrate in 50 mM HEPES, pH 7.6/0.5 mM EDTA/30 mM MgSO₄ (10 μM substrate for HL-60 cells and 20 μM substrate for U937 cells).

FIG. 11. Luminescence from H226 cells treated with lactacystin, 0.04% TMN-6 and 20 μM LLVY-aminoluciferin (SEQ ID NO:1) in 50 mM HEPES, pH 7.6/0.5 mM EDTA/30 mM MgSO₄ at 22° C.

FIGS. 12A-F. A) Effect of Mg concentration in a luminescent assay. Approximately 25,000 HL-60 cells were treated with 0.5 mM EDTA, 20 μg/ml digitonin and 20 μM substrate. B) Mg effect on half-life kinetics in a luminescent assay. Approximately 25,000 HL-60 cells were treated with 0.5 mM EDTA, 20 μg/ml digitonin and 20 μM substrate. C) Effect of Mg concentration in a luminescent assay with U937 cells. Cells were treated with 20 μg/ml digitonin and 20 μM substrate. D) Effect of Mg concentration in a luminescent assay with U937 cells. Cells were treated with 20 μg/ml digitonin and 20 μM substrate. E) Effect of Mg concentration in a fluorescent assay. HL-60 cells at 22° C. in the absence of lactacystin were contacted with 20 μg/ml digitonin, 20 μM LLVY-AMC (SEQ ID NO:1) in 50 mM HEPES, pH 7.6/0.5 mM EDTA, and various concentrations of MgSO₄. F) Luminescence versus Mg concentration. HL-60 cells at 22° C. in the absence of lactacystin were contacted with 20 μg/ml digitonin, 20 μM LLVY-aminoluciferin (SEQ ID NO:1) in 50 mM HEPES, pH 7.6/0.5 mM EDTA, and various concentrations of MgSO₄.

FIGS. 13A-C. A) Luminescence versus EDTA concentration. U937 cells at 22° C. in the absence of lactacystin were contacted with 20 μg/ml digitonin, 20 μM substrate, 30 mM MgSO₄ and various concentrations of EDTA. B) Luminescence versus EDTA concentration in U937 cells treated with lactacystin. Cells were contacted with 20 μg/ml digitonin, 20 μM substrate, 30 mM MgSO₄ and various concentrations of EDTA. Data is from a 30 minute time point. C) Luminescence versus EDTA concentration in PA-1 cells treated with lactacystin. Cells were contacted with 20 μg/ml digitonin, 20 μM substrate, and 30 mM MgSO₄. Data is from a 30 minute time point.

FIG. 14. Kinetics of a luminescent proteasome assay at varying pH. Approximately 25,000 Jurkat cells at 22° C. were contacted with 20 μM substrate in 50 mM HEPES/0.5 mM EDTA/30 mM MgSO₄.

FIG. 15. Effect of pH and substrate concentration in a luminescent proteasome assay. Approximately 25,000 Jurkat cells at 22° C. were contacted with varying amounts of substrate in 50 mM HEPES at pH 7.6 or 8.2/0.5 mM EDTA/30 mM MgSO₄.

FIG. 16. Profiles of three fluorophores which may be useful in multiplex assays. Ex=excitation spectra; em=emission spectra.

### DETAILED DESCRIPTION OF THE INVENTION

Definitions

As used herein, a “luminogenic assay” includes a reaction in which a first molecule, e.g., a peptide or polypeptide substrate for a first enzyme, the product of a reaction between the first molecule and an appropriate (first) protein, and/or a product of a reaction between a different protein and the product of the first reaction, is luminogenic. Thus, a luminogenic assay may directly or indirectly detect, e.g., measure, one or more activities of proteasomes or the amount or presence of a moiety other than proteasomes which moiety may be associated with an enzymatic reaction, e.g., is a cofactor for the reaction, a substrate for the reaction or the enzyme, or is a molecule which is bound by and/or altered by the moiety. Luminogenic assays include chemiluminescent and bioluminescent assays including but not limited to those which employ or detect luciferase, β-galactosidase, β-glucuronidase, β-lactamase, a protease, alkaline phosphatase, or peroxidase, and suitable corresponding substrates, e.g., modified forms of luciferin, coelenterazine, luminol, peptides or polypeptides, dioxetanes, dioxetanones, and related acridinium esters.

As used herein, a “luminogenic assay reagent” includes a substrate, as well as a cofactor(s) or other molecule(s) such as a protein, e.g., an enzyme, for a luminogenic reaction.

A “nonluminogenic assay” includes a reaction in which a first molecule, e.g., a protein (a peptide or polypeptide), a (first) product of a reaction between the first molecule and a suitable (first) protein (peptide or polypeptide), or a product of a reaction between a different protein and the first product is/are not luminogenic but may be otherwise detectable, e.g., the substrate and/or product(s) are detected using a fluorescent or calorimetric assay, which directly or indirectly measures one or more activities of a proteasome or the amount or presence of a moiety other than a proteasome, which moiety may be associated with an enzymatic reaction, e.g., the moiety is a cofactor, a substrate or an enzyme for the reaction such as another activity associated with the proteasome, or a molecule which interacts with the moiety.

As used herein, a “fluorogenic assay reagent” includes a substrate, as well as a cofactor(s) or other molecule(s), e.g., a protein, for a fluorogenic reaction.

As used herein, a “cell membrane permeabilization reagent” includes any reagent that when contacted with a cell partially disrupts the cell membrane allowing entry of a material into the cell that would normally be excluded from entry into live cells not exposed to the reagent, e.g., a reagent such as Trypan Blue, propidium iodine, or ethidium bromide.

As used herein, a “proteasome-specific proteolytic activity” is a trypsin-like, chymotrypsin-like or caspase-like activity, that is inhibitable by a proteasome-specific inhibitor, e.g., lactacystin.

### METHODS OF THE INVENTION

The invention provides a homogeneous assay to detect one or more activities of proteasomes in a permeabilized cell, including a multiplexed assay method to detect at least two different molecules or activities, one of which includes detecting proteasome activity either simultaneously or sequentially with other molecules or activities. For instance, one or more enzyme-mediated reactions are performed under conditions effective to convert at least one enzyme substrate to a product of a reaction between the substrate and the enzyme, and the product has a different characteristic (signal) from the substrate. For two enzyme-mediated reactions with two substrates, the reactions are performed concurrently or sequentially, under conditions effective to convert each substrate to a product of a reaction between the substrate and corresponding enzyme, where each substrate and/or product has a different characteristic. The resulting signal(s) is/are related to the activity, presence or amount of the molecule(s) to be detected. The luminogenic or fluorogenic methods of the invention employ luciferin, aminoluciferin, or a derivative thereof, or a fluorophore, which is modified to contain a substrate for an enzyme-mediated reaction, that is useful to detect the enzyme, a cofactor, an enzyme substrate, an enzyme inhibitor, and/or an enzyme activator for the enzymatic reaction.

In one embodiment, the methods according to the present invention provide a rapid, highly sensitive method for simultaneously or sequentially detecting multiple moieties or activities including proteasome activity in a single sample such as an aliquot of cells. In one embodiment, the method includes quantifying the presence or amount (activity) of a first enzyme, substrate or cofactor in a luminogenic assay and quantifying the presence or amount of a second enzyme, substrate or cofactor in a nonluminogenic assay, such as a fluorogenic assay. In one embodiment, reagents, e.g., substrates, for each reaction may be added together or sequentially. In another embodiment, the method includes quantifying the presence or amount of a first enzyme, substrate or cofactor in a fluorogenic assay and quantifying the presence or amount of a second enzyme, substrate or cofactor in a luminogenic assay. The intensity of the luminogenic or nonluminogenic signal is a function of the presence or amount of the respective molecule.

In one embodiment, to detect an enzyme of interest, the method employs at least two different reactions, where the first reaction is a nonluciferase enzyme-mediated reaction with a substrate for the enzyme of interest which yields a substrate for beetle luciferase, and the second reaction is a beetle luciferase-mediated reaction. Thus, a luminogenic assay may indirectly detect, e.g., measure, the amount, presence or specific activity of, for example, an enzyme, cofactor or substrate for a nonbeetle luciferase-mediated reaction, an inhibitor of the nonbeetle luciferase-mediated reaction, or an activator of the nonbeetle luciferase-mediated reaction. In those reactions, a modified luciferin, aminoluciferin or a derivative thereof, is employed which is a substrate for the nonbeetle luciferase enzyme, and the product of the reaction is a substrate for a beetle luciferase.

For a one step assay, a reaction mixture may contain reagents for two reactions, such as reagents for a nonbeetle luciferase enzyme-mediated reaction and a beetle luciferase-mediated reaction or a reaction mixture for a single reaction, e.g., for a reaction between a fluorophore modified to contain a substrate for an enzyme and the enzyme. For assays which employ at least two reactions, the order of adding the molecules for the assays can vary. If initiated and conducted sequentially (whether in the same vessel or not), adjustments to reaction conditions, e.g., reagent concentration, temperatures or additional reagents, may be performed. For instance, a quenching agent or enhancing agent may be added between reactions (see, e.g., U.S. Pat. Nos. 5,774,320 and 6,586,196, the disclosures of which are specifically incorporated by reference herein). In one embodiment, the two or more reactions are carried out simultaneously in a single reaction mixture. Optionally, the assays are a homogeneous assay, e.g., the components are mixed prior to adding the mixture to the sample. Results may be read without additional transfer of reagents.

Two general types of multiplexed assays are contemplated. In the first, multiple moieties, e.g., one or more enzymes, one or more substrates and/or one or more cofactors for an enzyme-mediated reaction, are assayed in the same reaction mixture. Each enzyme is capable of converting at least one of the substrates to a corresponding product, where the substrate(s) and/or corresponding product(s), or product(s) of a reaction between one of the corresponding products and another enzyme, have different detectable characteristics that allow the substrates and/or the products to be individually detected when present in the same reaction mixture. The order of adding the molecules for these assays can vary. Thus, individual reactions may be initiated and/or conducted simultaneously or sequentially. If initiated and conducted sequentially, the different detectable characteristics may require different detection methods, and/or adjustments to reaction conditions, e.g., reagent concentration, temperatures or additional reagents, may be performed. For instance, a quenching agent or enhancing agent may be added between reactions, as described above. In one preferred embodiment, the two or more reactions are carried out simultaneously in a single reaction mixture, where each of the enzymes is effective to convert one of the substrates in the reaction mixture to a product. This embodiment may be used, for example, to determine the activity, presence or amount of at least two different enzymes, substrates and/or cofactors in a cell, or at least two different enzymes, substrates or cofactors for two different reactions, at least one of which is detected in permeabilized cells and the other in intact cells, permeabilized cells, a cell lysate or cell supernatant. In addition, the reaction may contain one or more test agents, e.g., enzyme inhibitors or activators, and/or different concentrations of inhibitors, activators, or substrates. Optionally, the assays are employed as a homogeneous assay, e.g., the one or more substrates and additional components are mixed prior to adding the mixture to the sample. Results may be read without additional transfer of reagents.

In a second assay type, two or more enzyme-mediated reactions are carried out in tandem. The separate reactions may be performed at the same time or at different times. The reactions may contain one or more of the same or different enzymes, one or more of the same or different test agents, e.g., enzyme inhibitors or activators, and/or different concentrations of inhibitors, activators, or substrates. In one embodiment, each reaction mixture contains at least two substrates capable of being converted to a product, where the substrate(s) and/or corresponding product(s), and/or a product(s) of a reaction between the product of one of the enzyme/substrate pairs and a different enzyme, have different detectable characteristics.

The assays of the present invention thus allow the detection of multiple moieties including multiple enzymes or cofactors in a sample, e.g., a sample which includes eukaryotic cells, e.g., yeast, avian, plant, insect or mammalian cells, including but not limited to human, simian, murine, canine, bovine, equine, feline, ovine, caprine or swine cells, or cells from two or more different organisms, where at least proteasome activity is detected in permeabilized cells, and the activity, presence or amount of another molecule is detected in nonpermeabilized cells, supernatants of nonpermeabilized cells, permeabilized cells, cell lysates or a fraction of a cell lysate. The cells may not have been genetically modified via recombinant techniques (nonrecombinant cells), or may be recombinant cells which are transiently transfected with recombinant DNA and/or the genome of which is stably augmented with a recombinant DNA, or which genome has been modified to disrupt a gene, e.g., disrupt a promoter, intron or open reading frame, or replace one DNA fragment with another. The recombinant DNA or replacement DNA fragment may encode a molecule to be detected by the methods of the invention, a moiety which alters the level or activity of the molecule to be detected, and/or a gene product unrelated to the molecule or moiety that alters the level or activity of the molecule.

In one embodiment, the present invention relates to a method of measuring the presence or amount of one or more enzymes in a single aliquot of cells or a lysate thereof. In one embodiment, one of the enzymes is an endogenous protease found in the cytosol, such as a protease associated with proteasomes, and optionally another enzyme found in the cytosol or another location. For enzymes present in different cellular locations, such as a secreted protease and an intracellular cytosolic protease, a substrate for each enzyme can be added to a well with intact cells. The presence or amount of the secreted protease may be detected prior to detection of the cytosolic protease, which is detected after cell membrane permeabilization. In one embodiment, a non-cell permeant substrate for a cytosolic protease and a substrate for a secreted or released protease are added to a sample comprising cells and the cells are then permeabilized. Detection of the secreted or released protease may be before or after permeabilization. In another embodiment, a non-cell permeant substrate for a cytosolic protease or a secreted or released protease, and a cell permanent substrate for an intracellular enzyme are added to a sample comprising cells. The presence of the intracellular enzyme and the secreted or released protease may be detected without permeabilization. In one embodiment, the secreted or released protein is detected using fluorescence, luminescence or spectrophotometry.

The present methods can be employed to detect any moiety including any enzyme or any set of enzymes selected from any combination of enzymes including recombinant and endogenous (native) enzymes in addition to proteasome activity. In one embodiment, all of the enzymes to be detected are endogenous enzymes. In another embodiment, two enzymes to be detected are endogenous enzymes, one of which is associated with proteasomes and another enzyme is a recombinant enzyme. In another embodiment, one enzyme is an endogenous enzyme such as an activity associated with proteasomes and another enzyme is a recombinant enzyme. Other combinations apparent to one of ordinary skill in the art can be used in the present assays and methods according to the teachings herein. The enzymes include but are not limited to proteases, phosphatases, peroxidases, sulfatases, peptidases, and glycosidases. The enzymes may be from different groups based on the nature of the catalyzed reaction, groups including but not limited to hydrolases, oxidoreductases, lyases, transferases, isomerases, ligases, or synthases, or they may be from the same group so long as at least one of the enzymes has a partially overlapping or preferably a substantially different substrate specificity relative to at least one of the other enzymes. Of particular interest are classes of enzymes that have physiological significance. These enzymes include protein kinases, peptidases, esterases, protein phosphatases, isomerases, glycosylases, synthetases, proteases, dehydrogenases, oxidases, reductases, methylases and the like. Enzymes of interest include those involved in making or hydrolyzing esters, both organic and inorganic, glycosylating, and hydrolyzing amides. In any class, there may be further subdivisions, as in the kinases, where the kinase may be specific for phosphorylation of serine, threonine and/or tyrosine residues in peptides and proteins. Thus, the enzymes may be, for example, kinases from different functional groups of kinases, including cyclic nucleotide-regulated protein kinases, protein kinase C, kinases regulated by Ca²⁺/CaM, cyclin-dependent kinases, ERK/MAP kinases, and protein-tyrosine kinases. The kinase may be a protein kinase enzyme in a signaling pathway, effective to phosphorylate an oligopeptide substrate, such as ERK kinase, S6 kinase, IR kinase, P38 kinase, and AbI kinase. For these, the substrates can include an oligopeptide substrate. Other kinases of interest may include, for example, Src kinase, JNK, MAP kinase, cyclin-dependent kinases, P53 kinases, platelet-derived growth factor receptor, epidermal growth factor receptor, and MEK.

In particular, enzymes that are useful in the present invention include any protein that exhibits enzymatic activity, e.g., lipases, phospholipases, sulphatases, ureases, peptidases, proteases and esterases, including acid phosphatases, glucosidases, glucuronidases, galactosidases, carboxylesterases, and luciferases. In one embodiment, one of the enzymes is a hydrolytic enzyme. In another embodiment, at least two of the enzymes are hydrolytic enzymes. Examples of hydrolytic enzymes include alkaline and acid phosphatases, esterases, decarboxylases, phospholipase D, β-xylosidase, β-D-fucosidase, thioglucosidase, β-D-galactosidase, α-D-galactosidase, α-D-glucosidase, β-D-glucosidase, β-D-glucuronidase, α-D-mannosidase, β-D-mannosidase, β-D-fructofuranosidase, and β-D-glucosiduronase.

A substrate or cofactor for any particular enzyme-mediated reaction is known to those of skill in the art. Exemplary cleavage sites for some proteases are set forth in Table 1.

TABLE 1

| | |
|---|---|
| Protease | Cut Site(s) |

| | |
|---|---|
| Aminopeptidase M | Hydrolysis from free N-terminus |
| Carboxypeptidase Y | Hydrolysis from C-terminus |
| Caspase-1,4,5 | W/LEHD-X |
| | (SEQ ID NO:4) |
| Caspase-2,3,7 | DEXD-X |
| | (SEQ ID NO:5) |
| Caspase-6,8,9 | L/VEXD-X |
| | (SEQ ID NO:6) |
| Chymotrypsin | Y-X, F-X, T-X, |
| | (L-X, M-X, A-X, E-X) |
| Factor Xa | IEGR-X |
| | (SEQ ID NO:7) |
| Pepsin | F-Z, M-Z, L-Z, W-Z |
| | (where Z is a hydrophobic |
| | residue) but will cleave |
| | others |
| TEV | E(N)XYXQ-S/G |
| | (SEQ ID NO:8) |
| Thrombin | R-X |
| Trypsin | R-X, K-X |
| Tryptase | PRNK-X |
| | (SEQ ID NO:9) |
| β-secretase | EISEVK/NM/L-DAEFRHD |
| | (SEQ ID NO:10), e.g., |
| | SEVNL-DAEFR |
| | (SEQ ID NO:11) |

X is one or more amino acids

For alkaline phosphatase, it is preferable that the substrate includes a phosphate-containing dioxetane, such as 3-(2′-spiroadamantane)-4-methoxy-4-(3″-phosphoryloxy)phenyl-1,2-dioxetane, disodium salt, or disodium 3-(4-methoxyspiro[1,2-dioxetane-3,2′(5′-chloro)-tricyclo-[3.3.1.1^{3,7}]decan]-4-yl]phenyl phosphate, or disodium 2-chloro-5-(4-methoxyspiro{1,2-dioxetane-3,2′-(⁵′-chloro)-tricyclo{3.3.1.13,7]decan}-4-yl)-1-phenyl phosphate or disodium 2-chloro-5-(⁴-methoxyspiro{1,2-dioxetane-3,2′-tricyclo[3.3.1.13,7]decan}-4-yl)-1-phenzyl phosphate (AMPPD, CSPD, CDP-Star® and ADP-Star™, respectively).

For β-galactosidase, the substrate preferably includes a dioxetane containing galactosidase-cleavable or galactopyranoside groups. The luminescence in the assay results from the enzymatic cleavage of the sugar moiety from the dioxetane substrate. Examples of such substrates include 3-(2′-spiroadamantane)-4-methoxy-4-(3″-β-D-galactopyranosyl)phenyl-1,2-dioxetane(AMPGD), 3-(4-methoxyspiro[1,2-dioxetane-3,2′-(5′-chloro)tricyclo[3.3.1.1^{3,7}]-decan]-4-yl-phenyl-β-D-galactopyranoside (Galacton®), 5-chloro-3-(methoxyspiro[1,2-dioxetane-3,2′-(5′-chloro)tricyclo[3.3.1^{3,7}]decan-4-yl-phenyl-β-D-galactopyranoside (Galacton-Plus®), and 2-chloro-5-(4-methoxyspiro[1,2-dioxetane-3,2′(5′-chloro)-tricyclo-[3.3.1.1^{3,7}]decan]-4-yl)phenyl β-D-galactopyranoside (Galacton-Star®).

In assays for β-glucuronidase and β-glucosidase, the substrate includes a dioxetane containing β-glucuronidase-cleavable groups such as a glucuronide, e.g., sodium 3-(4-methoxyspiro{1,2-dioxetane-3,2′-(5′-chloro)-tricyclo[3.3.1.1^{3,7}]decan}-4-yl)phenyl-β-D-glucuronate (Glucuron™). In assays for a carboxyl esterase, the substrate includes a suitable ester group bound to the dioxetane. In assays for proteases and phospholipases, the substrate includes a suitable enzyme-cleavable group bound to the dioxetane.

For assays which include one dioxetane containing substrate, the substrate optionally contains a substituted or unsubstituted adamantyl group, a Y group which may be substituted or unsubstituted and an enzyme cleavable group. Examples of preferred dioxetanes include those mentioned above, e.g., those referred to as Galacton®, Galacton-Plus®, CDP-Star®, Glucuron™, AMPPD, Galacton-Star®, and ADP-Star™, as well as 3-(4-methoxyspiro{1,2-dioxetane-3,2′-(5′-chloro)-tricyclo[3.3.1.1^{3,7}]decan}-4-yl)phenyl-β-D-glucopyranoside (Glucon™), CSPD, disodium 3-chloro-5-(4-methoxyspiro{1,2-dioxetane-3,2′(5′-chloro)-tricyclo-[3.3.1.1^{3,7}]decan)-4-yl)-1-phenyl phosphate (CDP).

Substrates for other moieties, such as substrates for proteases not associated with proteasomes, may be modified with reporter molecules including but not limited to optic molecules such as fluorophores, an absorptive colored particle or a dye, radiolabels, enzymes such as a catalytic moiety that is effective to catalyze a detectable reaction in the presence of suitable reaction components, a subunit or fragment of an enzyme that is functional when associated with other subunit(s) or fragment(s), or a substrate for a subsequent reaction, e.g., one in which the product of that reaction is detectable.

Thus, both substrates for proteases associated with proteasomes and those for other moieties may be labeled with a fluorophore. As used herein, a “fluorophore” includes a molecule which is capable of absorbing energy at a wavelength range and releasing energy at a wavelength range other than the absorbance range. The term “excitation wavelength” refers to the range of wavelengths at which a fluorophore absorbs energy. The term “emission wavelength” refers to the range of wavelengths that the fluorophore releases energy or fluoresces.

One group of fluorescers is the xanthene dyes, which include the fluoresceins, rosamines and rhodamines. These compounds are commercially available with substituents on the phenyl group, which can be used as the site for bonding or as the bonding functionality. For example, amino and isothiocyanate substituted fluorescein compounds are available.

Another group of fluorescent compounds are the naphthylamines, having an amino group in the alpha or beta position, usually alpha position. Included among the naphthylamino compounds are 1-dimethylaminonaphthyl-5-sulfonate, 1-anilino-8-napththalene sulfonate and 2-p-toluidinyl-6-naphthalene sulfonate. Some naphthalene compounds are found to have some non-specific binding to protein, so that their use requires employing an assay medium where the amount of protein is minimized. Other fluorescers are multidentate ligands that include nitrogen-containing macrocycles, which have conjugated ring systems with pi-electrons. These macrocycles may be optionally substituted, including substitution on bridging carbons or on nitrogens. Suitable macrocycles include derivatives of porphyrins, azaporphyrins, corrins, sapphyrins and porphycenes and other like macrocycles, which contain electrons that are extensively delocalized. The azaporphyrin derivatives include phthalocyanine, benzotriazaporphyrin and naphthalocyanine and their derivatives.

In some instances fluorescent fusion proteins may be employed, e.g., a green, red or blue fluorescent protein or other fluorescent protein fused to a polypeptide substrate. In other embodiments, a fluorescent protein may itself be a substrate for a hydrolytic enzyme. A “fluorescent protein” is a full-length fluorescent protein or a fluorescent fragment thereof.

A non-limiting list of chemical fluorophores of use in the invention, along with their excitation and emission wavelengths, is shown in Table 2. Excitation and emission values can change depending on reaction conditions, such as pH, buffer system, or solvent.

TABLE 2

| Fluorophore | Excitation (nm) | Emission (nm) |
|---|---|---|
| Fluorescein (FITC) | 495 | 525 |
| Hoechst 33258 | 360 | 470 |
| R-Phycoerythrin (PE) | 488 | 578 |
| Rhodamine (TRITC) | 552 | 570 |
| Quantum Red ™ | 488 | 670 |
| Texas Red | 596 | 620 |
| Cy3 | 552 | 570 |
| Rhodamine-110 | 499 | 521 |
| AFC | 380 | 500 |
| AMC | 342 | 441 |
| Resorufin | 571 | 585 |
| BODIPY FL | 504 | 512 |
| BODIPY TR | 591 | 620 |

In one embodiment, one of the enzymes is detected using a substrate which includes an amino-modified luciferin or a carboxy protected derivative thereof, which modification includes a substrate for the enzyme. In one embodiment, the modification is one or more amino acid residues which include a recognition site for a protease. In one embodiment, a peptide with the recognition site is covalently linked to the amino group of aminoluciferin or a carboxy-modified derivative thereof via a peptide bond. In one embodiment, the N-terminus of a peptide or protein substrate is modified to prevent degradation by aminopeptidases, e.g., using an amino-terminal protecting group. In the absence of the appropriate enzyme or cofactor, a mixture including such a substrate and luciferase generates minimal light as minimal aminoluciferin is present. In the presence of the appropriate enzyme, the bond linking the substrate and aminoluciferin can be cleaved by the enzyme to yield aminoluciferin, a substrate for luciferase. Thus, in the presence of luciferase, for instance, a native, recombinant or mutant luciferase, and any cofactors and appropriate reaction conditions, light is generated, which is proportional to the presence or activity of the enzyme.

In one embodiment, one of the enzymes is detected using a substrate which includes a fluorophore. In one embodiment, the substrate includes one or more amino acid residues which include a recognition site for a protease. In one embodiment, the substrate is covalently linked to one or more fluorophores. In the absence of the appropriate enzyme or cofactor, a mixture including such a substrate generates minimal light at the emission wavelength as the fluorescent properties of the fluorophore are quenched, e.g., by the proximity of the quenching group such that the properties of a substrate-fluorophore conjugate are changed, resulting in altered, e.g., reduced, fluorescent properties for the conjugate relative to the fluorophore alone. In the presence of the appropriate enzyme, cleavage of the conjugate yields the fluorophore. In another embodiment, prior to cleavage, the conjugate is fluorescent but after cleavage with the enzyme, the product(s) have altered spectra.

In one embodiment, the conditions for at least two of the reactions are compatible. For instance, the conditions for at least 2 enzymes, and preferably the conditions for 3 or more enzymes, e.g., 4 or more enzymes, are compatible. A group of similar enzymes will generally have compatible reaction conditions, such as pH and ionic strength, however, cofactor requirements, metal ion requirements, and the like, involving assay components having relatively low mass concentrations, e.g., cofactors, need not be common. Common conditions include conditions such that each of the enzymes provides a measurable rate during the course of the reaction and will generally be that each of the enzymes has at least about 10%, usually at least about 20%, preferably at least about 50%, of its maximum turnover rate for the particular substrate, without significant interference from the components added for the other enzyme(s).

Alternatively, the conditions for one reaction may not be compatible with another reaction although substrates for both reactions are present. In such embodiments, one enzyme is active but cannot react with its substrate. In one embodiment, for example, where conditions for two reactions are not compatible, individual enzyme-assay reactions are carried out sequentially and/or in separate reaction mixtures. Following the enzyme assay, the reaction mixture (or a portion thereof) may be combined with another reaction. Each individual reaction mixture may contain one or more enzymes and one or more substrates. In its simplest form, a single enzyme to be assayed and a single substrate for that enzyme are in each reaction mixture. The set of substrates employed in the reaction has the same general properties as that required in the single-reaction multiplexed assay. That is, each substrate and/or corresponding product have unique characteristics, allowing them to be distinguished from one another.

The order of detection of molecules in the reactions can vary. In one embodiment, regardless of whether reactions are initiated at the same time or not, the molecule detected by a luminogenic assay is detected, then the molecule detected by the nonluminogenic assay is detected. Alternatively, regardless of whether reactions are initiated at the same time or not, the molecule detected by the nonluminogenic assay is detected, then the molecule detected by the luminogenic assay is detected. In other embodiments, the presence or amount of two or more molecules is detected essentially simultaneously. In one embodiment, the presence or activity of one molecule to be detected is substantially decreased prior to detecting the presence or activity of the second molecule, e.g., by waiting until the first signal has diminished, e.g., by at least 50%, or by adding a quenching agent for the first reaction. Thus, in some embodiments, one or more of the reactions are terminated, e.g., by inhibiting an enzyme for the reaction, prior to detection. Preferably, the signal produced by one assay does not substantially interfere with the quantification of the signal produced by at least one other assay.

Kits of the Invention

The present invention also provides kits for detecting the presence, amount or activity of one or more moieties including one or more peptides or proteins, molecules which bind to and/or are altered by the peptides or proteins, cofactors, nucleic acid or other molecules in a sample such as a sample including intact cells, a cell lysate, e.g., a lysate which is at least partially purified, and/or a cellular supernatant, where at least one moiety is detected in a permeabilized cell. Such a kit includes at least one reagent for quantifying at least one of the moieties, e.g., one or more peptides and/or proteins, molecules bound by and/or altered by the peptides and/or proteins, cofactors, or other molecules, such as a substrate for at least one enzyme and a cell membrane permeabilization reagent, or a substrate for at least one enzyme, a cell membrane permeabilization reagent, and a reagent to detect viability, e.g., Trypan Blue, or another molecule, e.g., a nucleic acid binding dye.

A cell membrane permeabilization reagent for use in the kits and the methods described herein is one which in an effective amount is capable of permeabilizing a eukaryotic cell without substantially disrupting intracellular membrane bound organelles or compartments in the cells, thus preserving proteasome activity and specificity for an appropriate protease substrate. In one embodiment, the cell membrane permeabilization reagent is selected from digitonin, saponin (Quillaja saponaria), streptolysin-O, detergents and/or surfactants. The amount of a particular cell membrane permeabilization reagent to be used is selected based upon the cell type being permeabilized, the desired rapidity of permeabilization, the magnitude of linearity with respect to cell number, and/or the culture medium to which the reagent is added. The amount may be determined by preparing various concentrations of a particular cell permeabilizing reagent and combining each concentration with a selected cell and assaying for activity that can be inhibited with highly specific proteasome inhibitors such as lactacystin and/or epoxomicin. Concentration ranges for permeabilization with digitonin or saponin include but are not limited to about 10 μg/ml to about 50 μg/ml, e.g., about 20 μg/ml to about 40 μg/ml, and for detergents and/or surfactants concentrations of about 0.05% to about 0.1%. Examples of suitable cell membrane permeabilization reagents include digitonin, saponin, Thesit®, Tergitol® TMN-6, Tergitol® NP-9, Triton X-100 and NonIdet-40. Examples of detergents or surfactants that were not compatible with a proteasome/luciferase reaction include SDS, CHAPS, TOMAH®, Tween®-20, Geropon® T-77, BioTerge and Brij-35. Preferred cell membrane permeabilization reagents are employed in an amount that does not substantially affect proteasome and/or luciferase activity.

To detect proteasome activity in a cell-based assay, cells are subjected to differential permeabilization through the use of a cell permeabilization reagent. For instance, low concentrations of digitonin (from Digitalis) or saponin (Quillaja bark) yield a fraction consisting of cytosolic proteins (Ramsby et al., 1994). Digitonin complexes with the cholesterol lipids of the plasma membrane, forms pores and allows release of cytosolic proteins. Low concentrations of Triton X-100 enrich for membrane and organelle proteins while maintaining nuclear integrity (Ramsby et al., 1994). Tween-40/deoxycholate lyses cells and destroys nuclear integrity and releases proteins loosely associated with the detergent resistant cytosol, and SDS or CHAPS lyse cells and extract the cytoskeleton, insoluble nuclear proteins and other hydrophobic proteins (Ramsby et al., 1994).

The invention will be further described by the following non-limiting examples. For all examples, suitable control reactions are readily designed by those skilled in the art.

### EXAMPLE I

### Protease Retention and Release Cell Viability Multiplex Assays

Live cell and dead cell assays are widely used to monitor the change in cellular viability in response to specific chemical, biological or physical treatments. Viability and cytotoxicity assays are generally converse and measure different biomarkers. Methods for assessment of general changes in cell viability by cytotoxicity have historically related to changes in outer membrane permeability. Classical methods of detecting compromised membrane structure include trypan blue exclusion, nucleic acid staining, and lactate dehydrogenase release (Riss et al., 2004; Myers et al., 1998). Assays for the assessment of cell function or proliferation include tritiated thymidine incorporation, ATP content, tetrazolium dye conversion or fluorescein diacetate (Cook et al., 1989). The assumption is that intact cell membranes do not allow bulky charged molecules or peptides to enter from the extracellular space into the cytosol. Conversely, damaged membranes allow free permeability of dyes or compounds into the cell, or cellular contents out of cells. This permeability phenomenon is the basis for both dye labeling (“vital” dyes, DNA intercalators or esterase modified fluoresceins) and LDH release assays. Whereas, the existing techniques for determining cellular viability remain as useful and cost efficient applications, they have a number of technical or practical drawbacks which limit their utility in high content, multiplexed or high throughput formats. For example, current measures of cellular membrane integrity by LDH release (CytoTox-ONE™) or dye reduction capacity (CellTiter-Blue™) cannot be paired (a means for normalizing the data) due to the shared resazurin substrate and overlapping Ex/Em spectra. Moreover, the colored resazurin substrate utilized in both assays limits 2^{nd} assay signal window intensity (and sensitivity) with other endpoint assay measures (color quenching), and the concentrations and formats are not optimized for second assay reagent pairing, e.g., limiting volumes).

Existing live/dead cell formats use carboxyfluorescein and an ethidium homodimer, the latter a known potent mutagen. That format requires washing and substitution of the cell culture medium. Moreover, carboxyfluorescein exhibits spontaneous hydrolysis in aqueous solutions and ethidium homodimer intercalation, which stains DNA, may interfere with downstream data normalization.

Cultured mammalian cells contain a rich milieu of proteases, esterases, lipases, and nucleases. For instance, the four general classes of proteases (aspartic, cysteine, serine, and metal-dependent) are represented and are associated with specific functions of homeostatic maintenance. These cytosolic, lysosomal and transmembrane bound proteases are involved in intracellular protein degradation, generation of immunogenic peptides, posttranslational modification, and cell division (Tran et al., 2002, Constam et al., 1995, Vinitsky et al., 1997). The activity of these enzymes is regulated by various mechanisms including specialized compartmentalization (Bond et al., 1987). In response to extreme stress, environmental adversity, or committed progression of the apoptotic program, a commensurate loss of compartmentalization and membrane integrity is observed (Syntichaki et al., 2003, Haunstetter et al., 1998). Therefore, the release of stable proteolytic mediators into the cell culture medium in in vitro cell models represents a potential surrogate for cell death. Conversely, cytoenzymological staining of retained proteolytic enzymes parallels the phenotypic observation of cell health. Together, such proteolytic activities may help ascertain the relative number of viable or compromised cells in a cell culture population, e.g., a “live/dead” assay.

For protease based live/dead cell assays, in one embodiment, one substrate (for dead cells) is a substrate for a relatively abundant, active and conserved protease that is stable and active at cytosolic pH, e.g., 7.0 to 7.2, and has a label with a spectrally distinct readout (R/O). Preferably, the kinetics of cleavage of that substrate parallels LDH release, and the conditions for activity do not include toxic or membrane altering agents, e.g., salts or thiols, and results in fast assay times. The other substrate (for live cells) is a substrate for a relatively abundant and conserved protease, is cell permeable for viable cells, and the protease is active in a viable cell cytosolic environment but unstable in extracellular environments. That substrate has a label with a spectrally distinct R/O and the cleavage reaction proceeds so as to result in fast assay times. The use of the two substrates in a nondestructive assay can detect undesirable proliferative events and, due to the use of complementary and independent surrogates at different spectra, can reduce erroneous conclusions and reduce errors due to cell clumping or pipetting errors since the viability versus cytotoxicity ratio is independent of cell number variability in that well.

A. Protease Release Assay Formats with AMC or R110 Fluorescence or Aminoluciferin Luminescence Reporters

HL-60 cells were two-fold serially diluted then either lysed by the addition Triton X to 0.2% final or maintained by the addition of vehicle. 1/10^{th} volume of 200 μM Ala-Ala-Phe-AMC substrate in 100 mM Na Acetate, pH 4.5, was added to the lysates or cells and incubated for an additional hour at 37° C. The fluorescence associated with lysed or viable cells was then measured at Ex. 360 Em. 460 using the CytoFluor II.

Jurkat cells undergoing active doubling were counted by trypan blue exclusion and found to be greater than 95% viable. The cells were adjusted to 100,000 cells/ml in RPMI 1640+10% FBS and split into two aliquots. One aliquot was sonicated using a Misonix 3000 equipped with a microtip at 30% power for 3×5 second pulses. The other fraction was incubated in a 37° C. water bath during the sonication procedure (about 5 minutes in total). The cell suspension and lysate fractions were then blended into varying viabilities by ratio mixing representing 0-100% viability. The blended cell samples were then added to a white-walled, clear-bottomed 96 well plate (Costar) in 100 μl volumes. (Ala-Ala-Phe)₂-R110 was diluted to 1000 μM in RPMI-1640 and added in 1/10^{th} volumes to the plate. The plate was incubated for 30 minutes before measuring fluorescence at Ex 485 Em 530 using a CytoFluor II.

Jurkat cells undergoing active doubling were counted by trypan blue exclusion and found to be greater than 95% viable. The cells were adjusted to 100,000 cells/ml in RPMI 1640+10% FBS and split into two aliquots. One aliquot was sonicated using a Misonix 3000 equipped with a microtip at 30% power for 3×5 second pulses. The other fraction was incubated in a 37° C. water bath during the sonication procedure (about 5 minutes in total). The cells solution and lysate fractions were then blended into varying viabilities by ratio mixing representing 0-100% viability. The blended cell samples were then added to a white-walled, clear-bottomed 96 well plate (Costar) in 100 μl volumes. The luminogenic protease release assay reagent was prepared by rehydrating a luciferin detection reagent cake (Promega V859A) with 10 ml of 10 mM Hepes, pH 7.5 and supplementing that reagent with Ala-Ala-Phe-aminoluciferin to 100 μM final concentration. 100 μl of the luminogenic protease release assay reagent was added to the wells of the plate and luminescence measured in kinetic mode using a BMG FLUOstar Optima.

The practical sensitivity of the AMC fluorescent format was calculated to be about 240 cells, a sensitivity value comparable to CytoTox-ONE™. The R110 format of the assay was similarly sensitive providing yet another fluorophore for multiplexing applications. Notably, the sensitivities from these assays were obtained without fluorescence quenching, a major obstacle for use of CytoTox-ONE™ or other resazurin-based assays in downstream multiplex applications. The exquisite linearity and range of the luminescent format allowed for statistical detection of as few as 200 cells in a population of 9800 viable cells. The non-lytic luminescent format offers another alternative for cytotoxicity detection.

B. Protease Release Assay Formats with Different Enzyme Targets

Actively doubling HL-60 cells were adjusted to 100,000 cells/ml and split into two aliquots. One aliquot was sonicated using a microtip Misonix 3000 with 30% power for three 5 second pulses. The other aliquot was held at 37° C. The cell suspension and lysates were then two-fold serially diluted in RPMI 1640+10% FBS in 100 μl volumes. Medium only served as the no cell control. A luciferin detection reagent cake (Promega V859A) was resuspended with 2.0 ml of 10 mM Hepes, pH 7.5. The luciferin detection reagent was then divided and made 1 mM with either Z-Leu-Leu-Val-Tyr-aminoluciferin or Ala-Ala-Phe-aminoluciferin. Each reagent was added to independent replicates of the plate in 1/10^{th} volumes and allowed to incubate for 15 minutes at 37° C. in the Me'Cour thermal jacketed water bath holder before luminescence measurement using the BMG FLUOstar Optima.

Although the Z-LLVY-aminoluciferin (SEQ ID NO:1) assay performed less optimally than the AAF-aminoluciferin sequence, it demonstrated that other proteases can be used as surrogates of compromised integrity. In this case, LLVY (SEQ ID NO:1) activity may be attributable to the chymotryptic activity of the proteasome.

C. Protease Release Time Course

HL-60 cells (25,000/well) were treated with 10 μM staurosporine or matched DMSO vehicle control over a 7 hour time course at 37° C. with 5% CO₂ in a clear bottomed, white walled 96-well plate (Costar). A 200 μM Ala-Ala-Phe-AMC substrate solution was created in 100 mM Na Acetate, pH 4.5. A 10 μl volume of the substrate ( 1/10^{th} volume of the sample) was added to the wells and incubated for an additional hour. “Protease release” activity was measured at Ex. 360 Em. 460 on a CytoFluor II. In a parallel set of wells, CytoTox-ONE™ reagent acted as the membrane integrity assay control. The reagent was added 10 minutes prior to measurement of fluorescence at Ex. 560 Em. 580.

The kinetics of cell permeability, i.e., LDH and protease release, mirrored each other and were consistent with the morphological observation of secondary necrosis in the cell populations. Presentation of the aminopeptidase substrate in an acidic Na Acetate formulation (final pH in sample about 6.5) was conducted to accommodate potential lysosomal protease activities.

D. Protease Release Activity pH Requirements

The pH requirement of the protease release activity was explored using 100 mM Na Acetate adjusted to pH 2.5, 3.5, and 4.5 and compared to non-adjusted culture medium (water vehicle). Ala-Ala-Phe-AMC was added to 200 μM in these buffers. A 1/10^{th} volume of the solutions was added to the plate and mixed briefly by orbital shaking. The plate was incubated for 40 minutes at 37° C., then fluorescence measured at Ex. 360 Em. 460 using the CytoFluor II.

Addition of 1/10^{th} volume of Na Acetate, pH 4.5 reduced the culture media to a final pH of about 6.5. The final pH of other lower pH solutions/medium combinations were not tested but previous experimentation suggested that adding 1/10^{th} volume of pH 2.5 Na Acetate reduced cell medium pH to about 5.5. It was found that the non-pH adjusted vehicle proved to be the most favorable for protease release activity. This activity is consistent with a cytosolic aminopeptidase and probably not a lysosomal protease (cathepsins etc.). This is significant because no detrimental or potentially cytotoxic adjuncts are required to measure protease release activity. This allows for more flexibility in the incubation time frame and is more amenable to a possible luminescence-based assay.

E. Protease Release Enzyme Subcellular Location

HL-60 cells were adjusted to 100,000 cells per ml and split into two aliquots. One aliquot was sonicated using a microtip Misonix 3000 with 30% power for three 5 second pulses. 100 μl of this lysate (confirmed morphologically) was added to multiple wells of a clear-bottomed, 96 well plate and two-fold serially diluted in RPMI 1640 with 10% FBS. Similarly, 100 μl of the non-sonicated cell suspension was added and serially diluted in multiple wells of the plate. NP-9 and digitonin were added to separate wells at 0.2% and 30 μg/ml final, respectively. An untreated control consisted of viable cells and a matched volume of water vehicle. A luciferin detection cake (Promega V859A) was rehydrated with 2 ml 10 mM Hepes, pH 7.5 and made 500 μM with Ala-Ala-Phe-aminoluciferin (Promega). 20 μl of this proluminescent protease release solution was added to all wells and luminescence measured after incubation at 37° C. for 15 minutes using a BMG FLUOstar Optima.

Sonication and NP-9, with the above parameters and concentrations, is known to disrupt not only the outer membrane, but also lysosomal contents (as measured by cathepsin release). Selective disruption by digitonin allows for trypan blue staining with no evidence of lysosomal rupture. Therefore, because the activities were similar between sonication or differential detergent lysis, and taken together with pH optima, one could surmise that the protease measured in the protease release assay is probably cytosolic and outside of an intact organelle(s).

F. Protease Release or Retention Enzyme Substrate Selectivity

Ala-Ala-Phe-AMC was obtained from Promega. Z-Leu-Leu-Val-Tyr-aminoluciferin (SEQ ID NO:1), Z-Leu-Arg-aminoluciferin, Z-Phe-Arg-aminoluciferin, Ala-Ala-Phe-aminoluciferin, (Ala-Ala-Phe)₂-R110 ((Ala-Ala-Phe)₂; SEQ ID NO:1), and (Gly-Phe)₂-R110 ((Gly-Phe)₂; SEQ ID NO:13) were synthesized by Promega Biosciences. Suc-Ala-Ala-Phe-AMC, H-Phe-AMC, H-Tyr-AMC, Glutyl-Ala-Ala-Phe-AMC (Glutyl-Ala-Ala-Phe; SEQ ID NO:14), H-Gly-Phe-AMC, Z-Gly-Ala-Met-AMC, Suc-Leu-Leu-Val-Tyr-AMC (SEQ ID NO:1), D-Ala-Leu-Lys-AMC, H-Gly-Ala-AMC, H-Gly-Gly-AMC, Suc-Ala-Ala-Phe-AMC, Z-Arg-Leu-Arg-Gly-Gly-AMC (Arg-Leu-Arg-Gly-Gly; SEQ ID NO:15), Z-Leu-Arg-Gly-Gly-AMC (Leu-Arg-Gly-Gly; SEQ ID NO:16) and Ac-Ala-Ala-Tyr-AMC were sourced from Bachem. Gly-Phe-AFC, Pro-Phe-Arg-AMC, Gly-Gly-Leu-AMC, and Ser-Tyr-AFC were obtained from Calbiochem. Z-Phe-Arg-AMC and Suc-Arg-Pro-Phe-His-Leu-Leu-Val-Tyr-AMC (Arg-Pro-Phe-His-Leu-Leu-Val-Tyr; SEQ ID NO:17) were purchased from Sigma.

All substrates were solubilized in DMSO from 10 to 100 mM depending upon inherent solubility. Fluorescent substrates were diluted to 100 μM to 1 mM in 10 mM Hepes, pH 7.5 or matched cell culture medium with 10% serum and added in 1/10^{th} volumes to lysed (freeze fractured, sonicated, or detergent) or untreated viable cells in white-walled, clear bottomed 96-well plates. HL-60 or Jurkat were used in the experimentation interchangeably because of their easily manipulated suspension phenotype. Plates were incubated for 15-30 minutes at 37° C. prior to measuring fluorescence by the CytoFluor II.

Luminescent substrates were added to a luciferin detection cake (Promega V859A) resuspended in 2 ml 10 mM Hepes, pH 7.5 to 500 μM. ⅕^{th} volume of the proluminescent reaction mixes were added to lysed (freeze fractured, sonicated, or detergent) or untreated viable cells in white-walled, clear bottomed 96-well plates. Again, HL-60 or Jurkat were used in the experimentation interchangeably. Plates were incubated at 37° C. in a MeCour' circulating heat block controlled by a Caron 2050 W exchange unit. Luminescence was measured between 15 and 30 minutes (signal steady state).

A broad variety of proteolytic substrates were examined in an effort to characterize potential substrate preferences for protease release or retention in compromised or viable cells (see Table 3). Amino-terminally blocked substrates (Z, Suc-, or Ac-) were chosen to delineate whether an endo or exopeptidase activity predominated. Non-blocked substrates (H- and the like) were examined to include the contribution of aminopeptidase activities. From this panel, at least three proteolytic profiles emerged: an aminopeptidase-like activity preferring unblocked Ala-Ala-Phe tripeptide, a proteosomal (chymotrypsin-like) activity measured by release of blocked Leu-Leu-Val-Tyr (SEQ ID NO:1) peptides, and an exceedingly labile activity by Gly-Phe, Gly-Ala, Phe-, Tyr- or Gly-Gly-Leu substrates. The latter activities were only measurable in viable, intact cells. Of further significance is that several fluorophores or proluminescent labels can be used to detect these activities, ultimately allowing for enhanced downstream multiplexing flexibility.

TABLE 3

| | | Reten- | |
|---|---|---|---|
| Substrate: | Target Protease(s) | tion | Release |
| Z-Phe-Arg-AMC | Cathepsin B, L | None¹ | None |
| Z-Gly-Gly-Leu-AMC | 20S Proteasome | ++* | None |
| Z-Arg-Leu-Arg-Gly- | Isopeptidase T | None | None |
| Gly-AMC | | | |
| (SEQ ID NO:15) | | | |
| Z-Leu-Arg-Gly-Gly-AMC | Isopeptidase T | None | None |
| (SEQ ID NO:16) | | | |
| S-R-P-F-H-L-L-V-Y-AMC | Proteasome, | None | None |
| (SEQ ID NO:17) | Chymotrypsin | | |
| H-Pro-Phe-Arg-AMC | Kallikrein | None | None |
| H-Gly-Gly-AMC | Aminopeptidase | None | None |
| H-Gly-Ala-AMC | Aminopeptidase | ++ | None |
| H-D-Ala-Leu-Lys-AMC | Plasmin | None | None |
| Ala-Ala-Phe-AMC | Tripeptidyl | None | ++++ |
| | Peptidase II | | |
| (Ala-Ala-Phe) 2 R110 | Tripeptidyl | None | ++++ |
| (SEQ ID NO:12) | Peptidase II | | |
| Ala-Ala-Phe-Aminoluc | Tripeptidyl | None | ++++ |
| | Peptidase II | | |
| Gluty-Ala-Ala-Phe-AMC | Chymotrypsin | None | None |
| (SEQ ID NO:14) | | | |
| Gly-Phe-AFC | Cathepsin C | ++++ | None |
| Gly-Phe-AMC | Cathepsin C | ++ | None |
| (Gly-Phe)2 R110 | Cathepsin C | None | None |
| (SEQ ID NO:13) | | | |
| Suc-Leu-Leu-Val-Tyr- | Calpain, | None | + |
| AMC | Chymotrypsin | | |
| (SEQ ID NO:1) | | | |
| Z-Leu-Leu-Val-Tyr- | Calpain, | None | ++ |
| Aluc | Chymotrypsin | | |
| (SEQ ID NO:1) | | | |
| Z-Gly-Ala-Met-AMC | | None | None |
| Ac-Ala-Ala-Tyr-AMC | Chymotrypsin | None | None |
| Z-Leu-Arg-Aluc | Cathepsin K | None | None |
| Z-Phe-Arg-Aluc | Cathepsin B, L | None | None |
| Ser-Tyr-AFC | Aminopeptidase | None | None |
| H-Phe-AMC | Aminopeptidase | +++ | None |
| | M | | |
| H-Tyr-AMC | ApM or Cathep- | ++ | None |
| | sin H | | |
| Suc-Ala-Ala-Phe-AMC | Chymotrypsin | None | None |

None denotes no statistical activity above control population.
(+) to (+++++) denotes the range of activity above control population from modest to robust

G. Multiplexed Protease Release and Retention Assays

1. Jurkat Dose Response

Actively doubling Jurkat cells were seeded into 96-well plates at a cell density of 20,000 cells per well in 50 μl volumes. Serial dilutions of the apoptosis inducing ligand, rTRAIL in RPMI 1640, were added to replicate wells from 250 ng to 244 pg/ml final concentration in an additional 50 μl volume. RPMI only served as uninduced control. The plate was incubated at 37° C. in 5% CO₂ for a period of 4 hours. Gly-Phe-AFC and Ala-Ala-Phe-AMC were simultaneously diluted to 1 mM in RPMI and added in a 1/10^{th} volume to the plate and were incubated for an additional 30 minutes at 37° C. Resulting fluorescence was measured at Ex 360 Em 460 and Ex 405 Em 530 using the CytoFluor II. After fluorescence measurements were completed, CellTiter-Glo® was added to wells in an equal addition and luminescence measured using the BMG FLUOstar Optima.

Two independent non-destructive surrogates of cell health (protease release and retention) were multiplexed to report population viability in a micro-titer plate format (see PCT/US2005/002158, which is incorporated by reference herein). The resulting data are converse measures of the health of that cell population. This relationship allows for use of a control and provides a level of normalization. Furthermore, a third measure of viability (ATP content) can be added in a sequential multiplex format with no interference or quenching allowing for further confidence in the interpretation of the data.

2. SK-MEL-28 and ACHN Cells

SK-MEL-28 or ACHN cells were seeded into white-walled, clear bottomed 96 well plates at a density of 10,000 cells per well in 100 μl volumes and allowed to attach at 37° C. in 5% CO₂ for a period of 2 hours. After attachment, 50 μl of medium was carefully removed and replaced with serial dilutions of either ionomycin or staurosporine in MEM+10% FBS. Medium only served as control. The plate was incubated for an additional 5 hours. A 1 mM solution of Gly-Phe-AFC was made in MEM and added to the wells in a 1/10^{th} volume. Resulting fluorescence was measured using a CytoFluor II. Caspase-Glo™ 3/7 reagent was then added and luminescence measure using a BMG FLUOstar Optima.

The protease retention substrate reported the general viability in the well, whereas the caspase specific reagents reported specific pathways of cytotoxicity. In this regard, caspase activation (and therefore apoptosis induction) is evident with staurosporine on SK-MEL-28, whereas ionomycin imitates a necrotic-type profile. An apoptotic profile is also observed with staurosporine treated ACHN.

3. HeLa Cells and Tamoxifen Treatment

HeLa cells were seeded into white-walled, clear bottomed 96 well plates at a density of 10,000 cells per well in 100 μl volumes and allowed to attach at 37° C. in 5% CO₂ for a period of 2 hours. After attachment, 50 μl of medium was carefully removed at 24, 7, 5, 3, 1 and 0 hours of exposure time and replaced with 50 μM tamoxifen in MEM+10% FBS. Medium only served as control. A protease retention and release reagent was prepared by rehydrating a luciferin detection reagent cake with 2 ml of 10 mM Hepes, pH 7.5. The solution was then made 500 μM with both Ala-Ala-Phe-aminoluciferin and Gly-Phe-AFC. A ⅕^{th} volume of the solution was added to all wells and incubated for 15 minutes at 37° C. in the Me'Cour thermo unit. Luminescence was measured by a BMG FLUOstar Optima and fluorescence measured using a CytoFluor II.

This example demonstrates that a mixed platform (fluorescence and luminescence) is possible in a configured protease retention and release assay. It is notable that these reagents are non-lytic and apparently non-toxic suggesting that they are amenable to other downstream applications that are spectrally distinct such as caspase-3/7 detection by the Apo-ONE™ assay.

4. Use of a Live/Dead Protease Assay with a DNA Stain

HeLa or HepG2 cells were seeded into white-walled, clear bottomed 96 well plates at a density of 10,000 cells per well in 100 μl volumes and allowed to attach at 37° C. in 5% CO₂ for a period of 2 hours. After attachment, 50 μl of medium was carefully removed and replaced with serial dilutions of tamoxifen or Ionomycin in MEM+10% FBS. Medium only served as control. Incubation with the compounds was continued for an additional 5 hours. A protease retention and release reagent was prepared by rehydrating a luciferin detection reagent cake (Promega V859A) with 2 ml of 10 mM Hepes, pH 7.5. The solution was then made 500 μM with both Ala-Ala-Phe-aminoluciferin and Gly-Phe-AFC. A ⅕^{th} volume of the solution was added to all wells and incubated for 15 minutes at 37° C. in the Me'Cour thermo unit. Luminescence was measured by a BMG FLUOstar Optima and fluorescence measured using a CytoFluor II. Next, remaining viable cells were lysed by the addition of 0.4% NP-9 detergent. After brief mixing on an orbital shaker, a 1:20 dilution of PicoGreen® (Molecular Probes) in MEM was added in an additional 1/10^{th} volume. Fluorescence associated with DNA/dye binding was measured using a CytoFluor II at Ex. 485 Em. 530.

This experiment not only expands the utility of protease based viability testing to two additional adherent cell types of screening favor, but incorporates a “total” measure by DNA staining. Because of spectral distinctness and mixed platform readout, all measures are non-interfering and non-quenching.

Discussion

Both drug discovery and primary research efforts continue to utilize increasingly sophisticated cell model systems. The obligate need to measure cell number and viability in these in vitro systems after experimental manipulation is well appreciated. This requirement is necessary to verify the validity of measures and normalize these responses within the context of complex biological systems.

Unfortunately, current chemistries for defining cellular viability and cytotoxicity have not kept pace with the new methodologies and techniques of biological inquiry and have therefore limited experimental options. For instance, the emergence of assay multiplexing, i.e., combination assays in the same well, have necessitated the requirement for compatible and spectrally distinct assay combinations without significant reductions in assay performance. This mandate is particularly important in regards to coupling general complimentary measures of cell health with a more specific event such as caspase activation or reporter gene modulation.

The aforementioned methodology for measurement of cell viability and/or cytotoxicity reporters that are compatible with many downstream assay applications. This is accomplished either by distinct fluorphores with divergent excitation and emission spectra or by integrating other reporter platforms such as luminescence. It is noteworthy that this is accomplished in a non-lytic and presumably non-toxic environment allowing for flexibility in assay windows for endpoint determinations. Furthermore, this technology is sufficiently sensitive and cost effective to accommodate throughput, miniaturization and automation. A comparison of advantages offered by various assays is provided in Table 4.

TABLE 4

| | Protease | Dye | | | Profluorecein | Radiological | |
|---|---|---|---|---|---|---|---|
| | Release | Exclusion | | | and | Incorporation | |
| Assay | and | (Trypan | Resazurin | LDH | Propidium | Or | |
| Attributes | Retention | Blue) | Reduction | Release | Iodide | Release | ATP |
| Homogeneous | Yes | yes | yes | yes | yes/no | no | yes |
| Non- | Yes | yes | yes | yes | yes | yes | no |
| Destructive | | | | | | | |
| Reagent | Yes | yes | yes | yes | no | yes | N/A |
| Stable in | | | | | | | |
| Culture | | | | | | | |
| Environment | | | | | | | |
| Non-toxic, | Yes | yes | yes | yes | no | no | yes |
| easy disposal | | | | | | | |
| Non-color | Yes | no | no | no | yes | yes | yes |
| quenching | | | | | | | |
| Fluorescence | Yes | no | yes | yes | yes | no | no |
| Luminescence | Yes | no | no | no | no | no | yes |
| Platform | Yes | no | no | no | no | no | no |
| Choice | | | | | | | |
| Compatible w | Yes | yes | yes | yes | yes* | no | no |
| Endpoint | | | | | (If spectrally | | |
| Multiplexes | | | | | distinct) | | |
| Ratioimetric | Yes | no | no | no | yes | no | no |
| normalization | | | | | | | |
| of | | | | | | | |
| response | | | | | | | |

In conclusion, to date, the balance of published effort in the study of mammalian proteases has revolved primarily around those either easily purified, secreted, or both. Whereas the information provided from these studies has provided insight into proteolytic mechanism, structure and function, little is known about other proteases other than what has been speculated from proteomic prediction.

Increasing evidence suggest that a number of cytosolic proteases are involved in mechanisms of cellular homeostasis. Although proteasomes are clearly involved in the liberation of cytosolic peptides, several findings suggest a role for other conserved cytosolic proteases (Vititsky et al., 1997; Constam et al., 1995).

The individual protease assays and the protease based live/dead cell assays described herein are more flexible for multiplexing due to spectral distinctness, allowing for assay complementarity or other endpoint assay combinations, e.g., AMC, AFC, R110, cresyl violet or luminescence, no dye quenching, no restrictive volumes, no retroengineering of assay chemistry, short incubation times, similar or better practical sensitivities (percent change in cell viability in a screening environment), no downstream interference with DNA binding assays, and no need for washing or centrifugation, e.g., homogeneous assays. Further, the substrates for proteases may be relatively simple, e.g., di or tri-peptides, are coupled to fluors or luminogenic substrates by well known chemistries, nontoxic and/or nonmutagenic, stable, and can be provided in various formats, e.g., in DMSO or dry.

### EXAMPLE II

### Cell-Based Assays for Proteasome Activities

Materials and Methods

Plate Preparation

NCI-H226 cells (ATCC #CRL-5826) were maintained as an attached line using RPMI 1640 (Sigma #R-8005) with 10% fetal bovine serum (Hyclone # SH30070) and passaged as needed. Jurkat, HL-60 and U937 suspension cell lines were likewise maintained and passaged. To prepare cells for a cell-based proteasome assay, adherent cells were harvested from the parent flask by removing medium, rinsing the flask with D-PBS and incubated with trypsin-EDTA (Sigma T-4040) for 3 to 4 minutes at 37° C. The trypsin reaction was stopped by adding complete medium containing serum, and cells were then centrifuged 4 minutes at 200×G. The cell pellet was suspended in fresh medium, cells counted by trypan blue exclusion and adjusted to 1.11×10⁵ cells/ml. A 96-well clear bottom/white walled plate (Costar# 3610) was obtained and 90 μl/well of cell suspension or medium alone was dispensed. Plate was cultured in a humidified 5% CO₂ incubator at 37° C. overnight for adherent cells. Suspension cells were equilibrated for about 1.5 hours prior to drug treatment.

Drug Addition

Lactacystin (Calbiochem # 03-34-0051) was initially suspended to 5 mM in water and used to prepare concentrated dilutions for addition to cells. 10×concentrated dilutions of lactacystin were prepared into culture medium so that the final concentration after addition of 10 μl/well ranged from 0 to 25 μM. Serial dilutions were prepared to achieve a series of intermediate dilutions. Stocks of other inhibitors, such as calpeptin and AdaAhx₃L₃VS (adamantane-acetyl-(6-aminohexanoyl)₃-(leucinyl)₃-vinyl-(methyl)-sulfone), were likewise prepared and diluted. For cell-based inhibition, drug was added to wells (10 μl/well) and the plate was then gently mixed by shaking for 60 seconds on a plate shaker. Plate was returned to the 37° C. incubator for 1.5 hours to allow the drug to enter cells.

2×Reagent Preparation

Reagent was prepared as follows: Luciferin detection reagent (Promega # V859A) was suspended with
100 mM HEPES (pH 7.6, adjusted using KOH) (Sigma H4034)
1 mM EDTA (Sigma)
60 mM MgSO₄ (Fisher Scientific # M63-500)
40 μg/ml digitonin (Sigma D-141)

The luciferin detection reagent (LDR) contains the following when reconstituted:
0.6% Prionex (Pentaphama, Basel, Switzerland)
0.4 mM ATP
100 μg/ml recombinant luciferase (Promega E140X)
2 U/ml of inorganic pyrophosphatase

For instance, one vial of V859A (Luciferin Detection Reagent) is suspended with 10 ml of buffer to which 50 to 100 μl of substrate is added, and 100 μl of cells are combined with 100 μl of substrate containing reagent. For aminoluciferin based substrates, a preincubation step may be employed. For example, 40 μM or 80 μM of Suc-LLVY-aminoluciferin (Promega; SEQ ID NO:1) protease substrate was added to the reconstituted LDR and incubated at 22° C. for 30 minutes. This pre-incubation depletes the free aminoluciferin present in the substrate, thereby reducing potential background luminescence.

Additions to Cells

A cell culture plate containing cells treated for 1 to 2 hours, e.g., 1.5 hours, with various concentrations of lactacystin or other inhibitor was removed from the incubator and equilibrated at 22° C. for 30 minutes to allow the contents to equilibrate uniformly. An equal volume (100 μl/well) of reagent was added to each well, and the plate was mixed using a orbital plate shaker for 1 minute. The assay plate was then maintained at 22° C. using a water bath. For luminescent read outs, luminescence was determined over time using a DYNEX@MLX luminometer, with the plate returned to the 22° C. water bath after each read to maintain a constant temperature.

Results

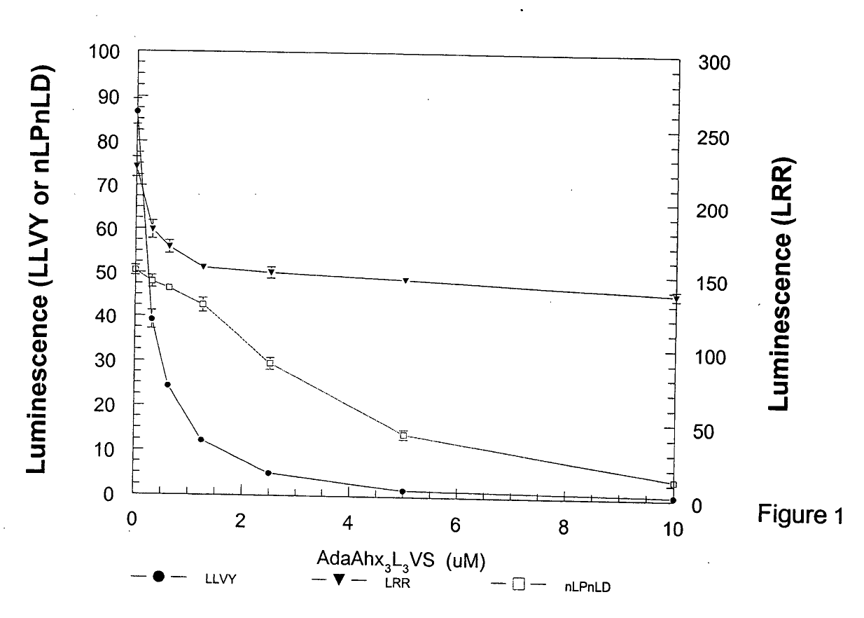

Luminogenic substrates were used to detect the chymoptrypsin-like (LLVY; SEQ ID NO:1), trypsin-like (LRR) and caspase-like (nLPnLD; SEQ ID NO:2) activities of the proteasome following treatment with various amounts of a cell permeable proteasome inhibitor which is potent against those activities (AdaAhx₃L₃VS; FIG. 1). The cells were cultured for 1.5 hours to allow the inhibitor to enter the cells and bind to proteasomes. Short incubations (for 1 to 2 hours) were found to be not toxic to the cells, although longer incubation times induced apoptosis.

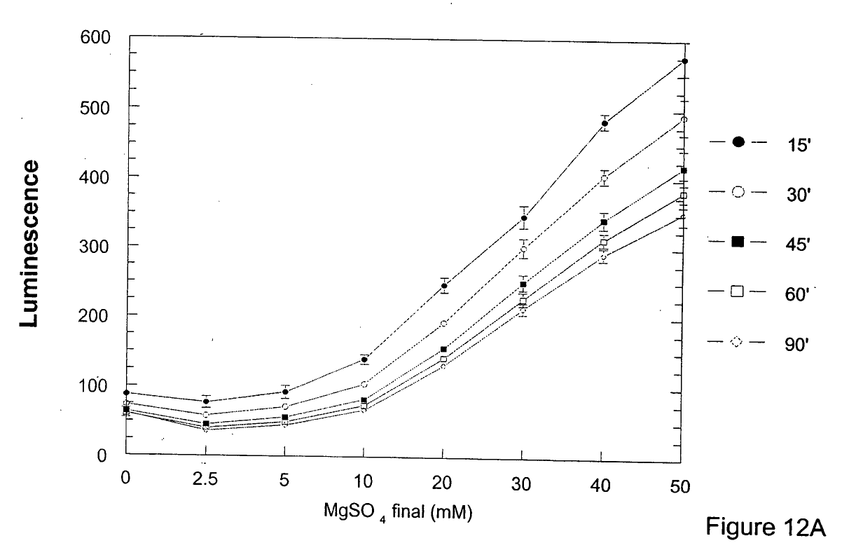

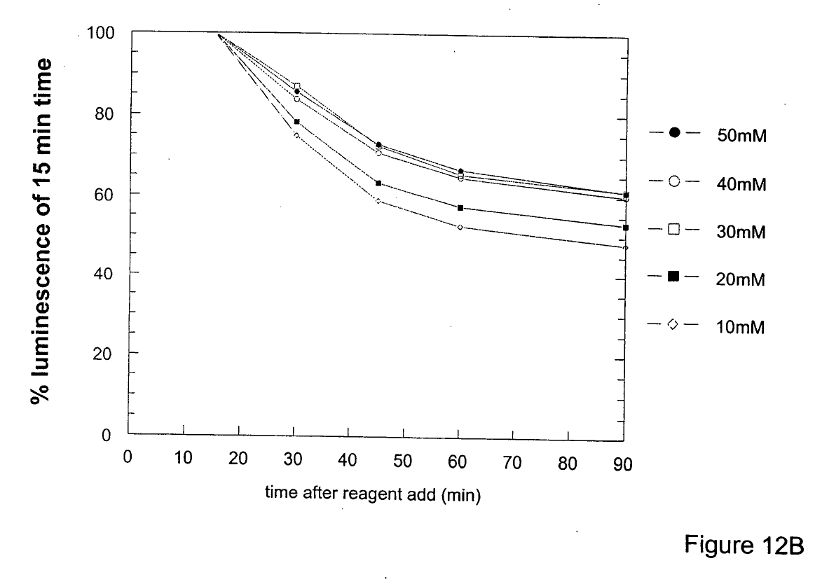

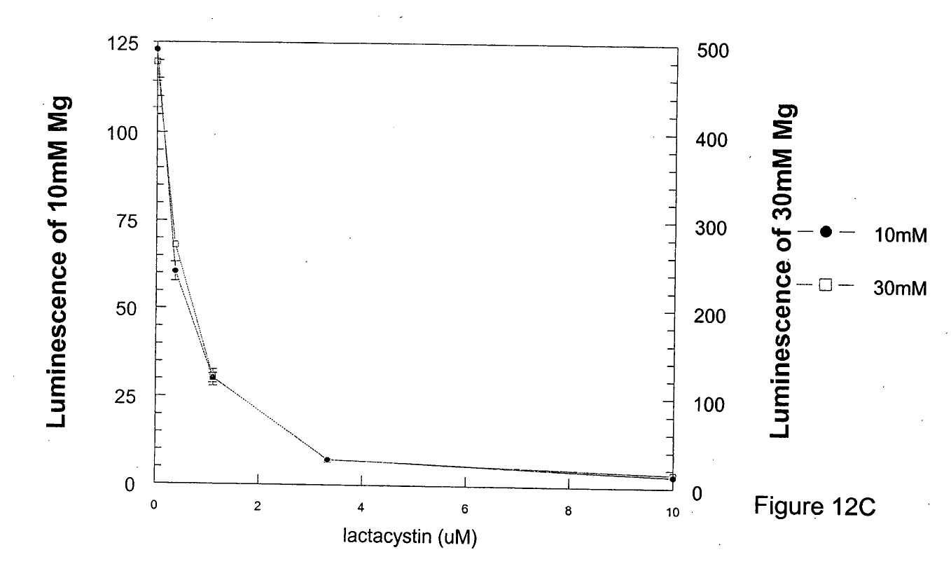

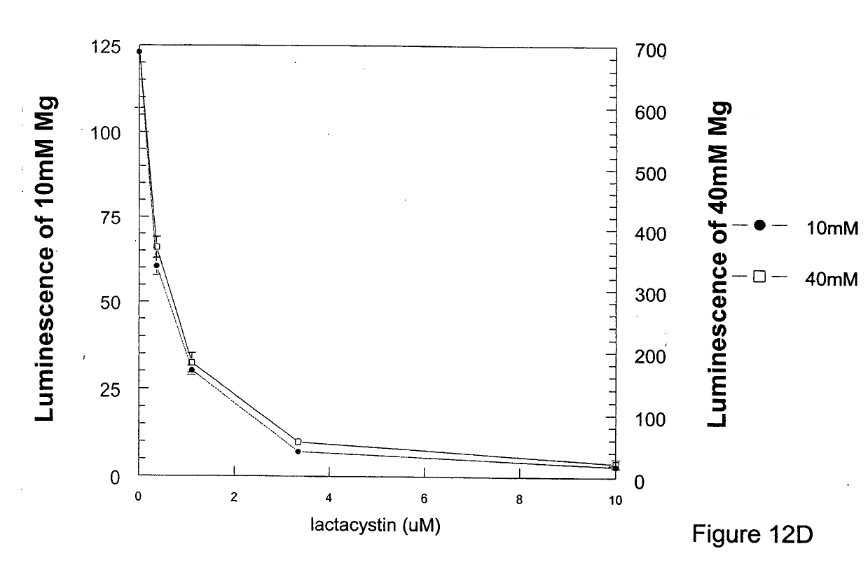

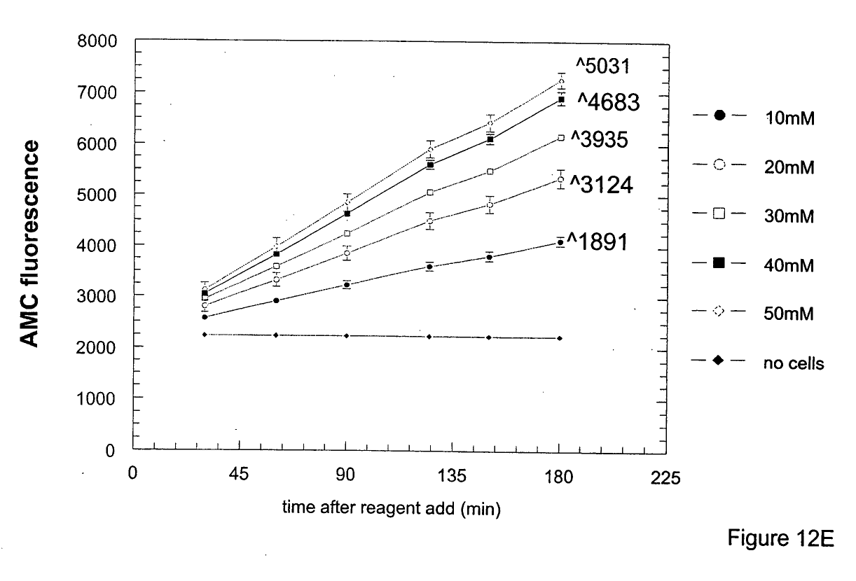

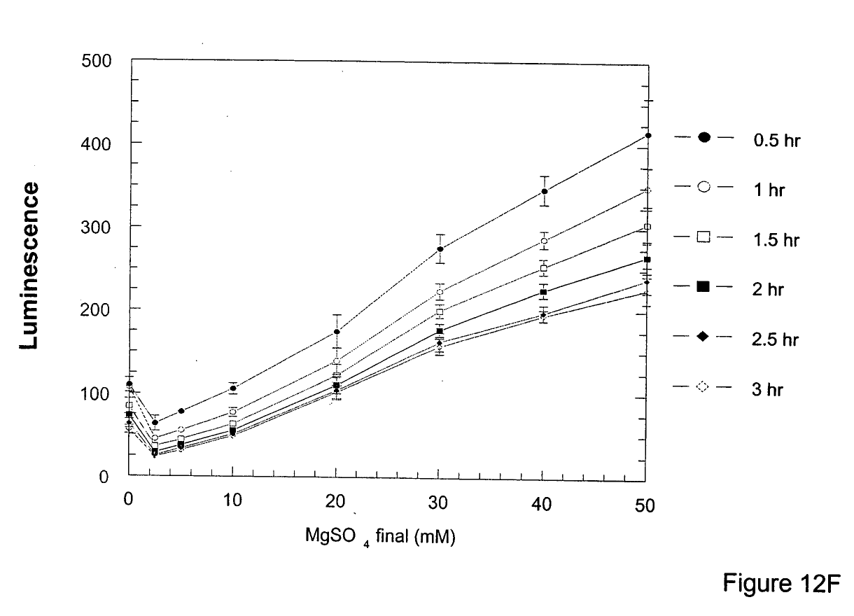

Substrates were added to a reaction mixture for a beetle luciferase-mediated reaction along with digitonin. The concentration of digitonin was chosen to selectively permeabilize the plasma membrane, allowing access to cytosolic molecules, without disturbing other organelles, particularly the lysosomes, which would release a pool of proteases. Although selective permeabilizations are typically done under serum-free conditions following medium removal to minimize serum interference with digitonin, as described below, selective permeabilizations may also be conducted in medium containing serum. Moreover, the efficiency of digitonin extraction is improved by EDTA, which may also help minimize the activity of other proteases, particularly calpain (see FIG. 12). The results show that the LLVY (SEQ ID NO:1) substrate had a wide dynamic range.

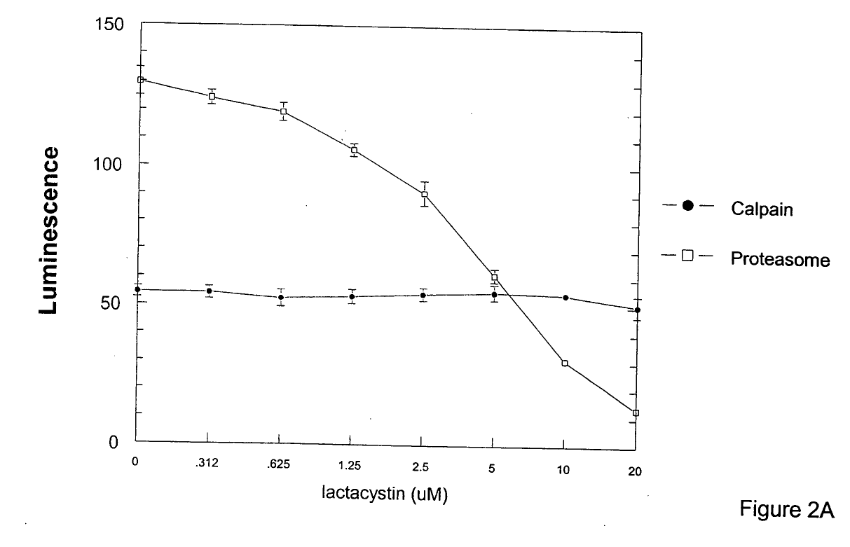

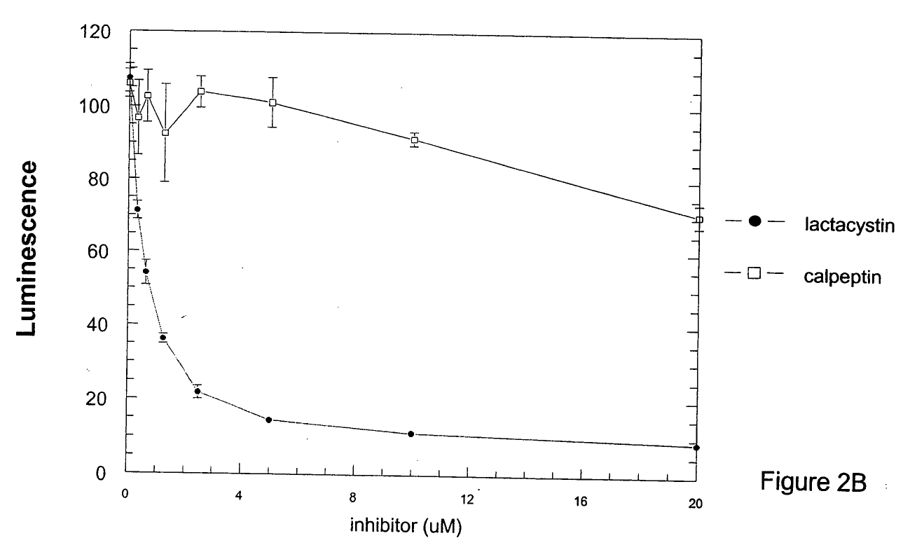

Lactacystin is a Streptomyces metabolite that covalently binds and modifies the highly conserved amino-terminal threonine of the mammalian proteasome subunit X (MB1) (Mellgren, 1997; Fenteany et al., 1995). The effect of lactacystin on purified calpain I is shown in FIG. 2A. The results show that lactacystin has a minimal effect on calpain I, which requires calcium to be active. The effect of a calpain I inhibitor, calpeptin, on proteasome activity in HL-60 cells is shown in FIG. 2B. Calpeptin, in contrast to lactacystin, had a minimal effect on proteasome activity after a 1.5 hour treatment. The inclusion of DTT (to aid the activity of calpain I) resulted in incomplete inhibition of the proteasome at 20 μM.

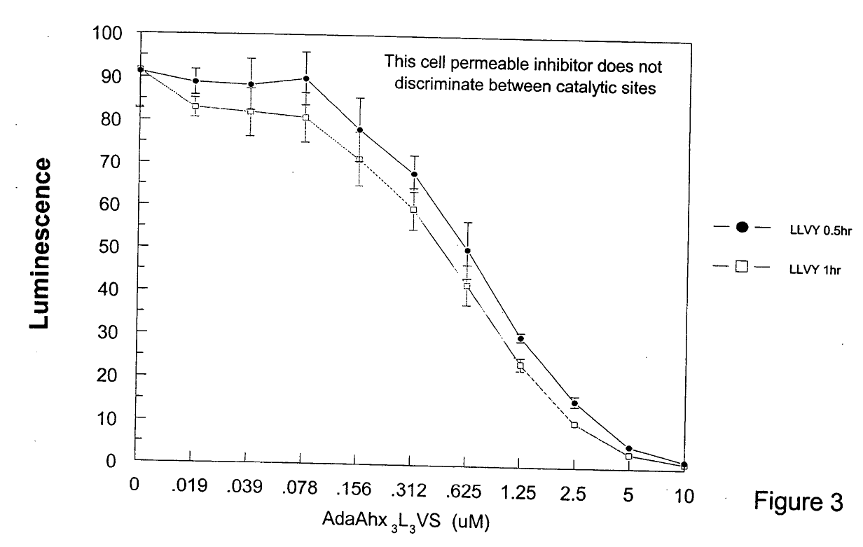

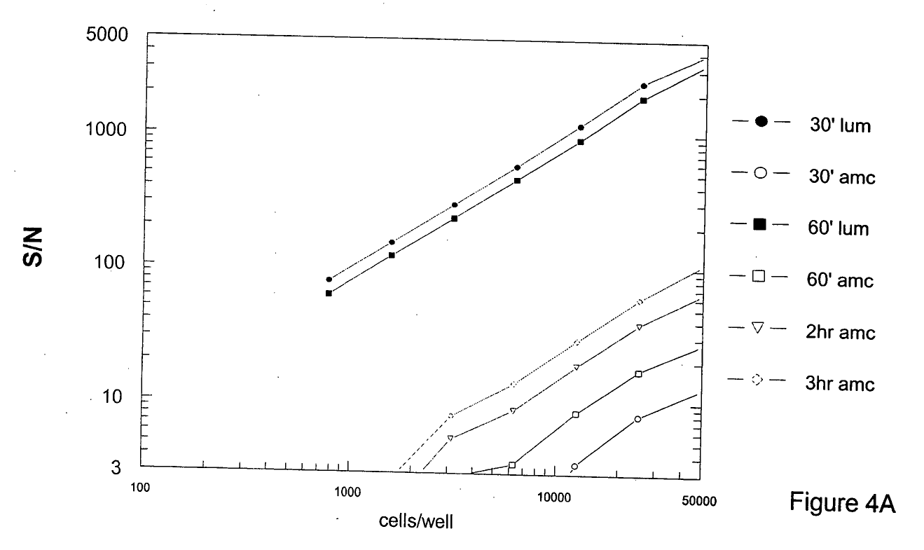

LLVY (SEQ ID NO:1) activity in Jurkat cells after AdaAhx₃L₃VS treatment is shown in FIG. 3. AdaAhx₃L₃VS blocked all three activities, however, the only results shown are for LLVY (SEQ ID NO:1) activity.

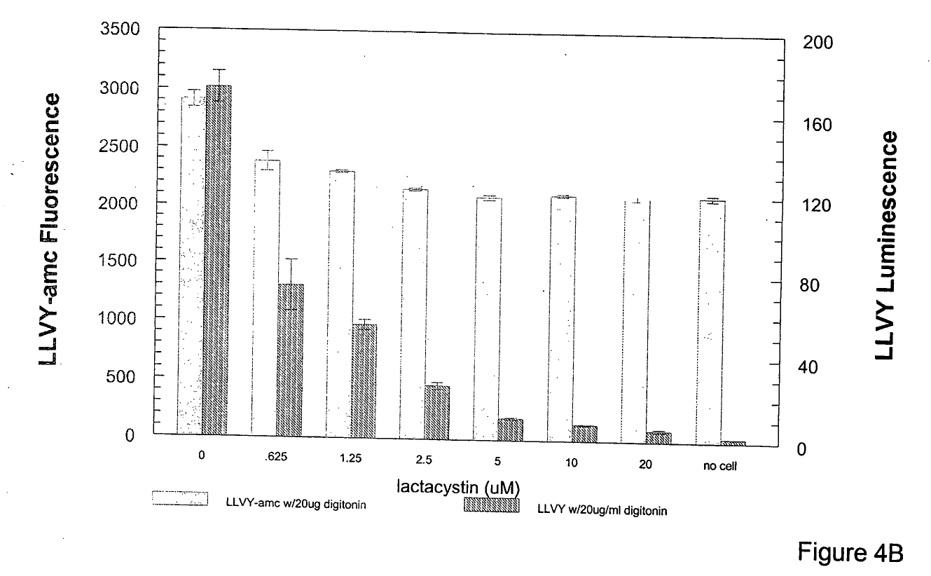

Luminescent and fluorescent assays (FIGS. 4A-B) with an aminoluciferin-LLVY (SEQ ID NO:1) substrate and an LLVY-AMC (SEQ ID NO:1) substrate, respectively, indicated that maximum luminescent sensitivity was reached at approximately 10 to 15 minutes, with the signal declining after that, while fluorescent sensitivity increased for periods up to 1 to 3 hours.

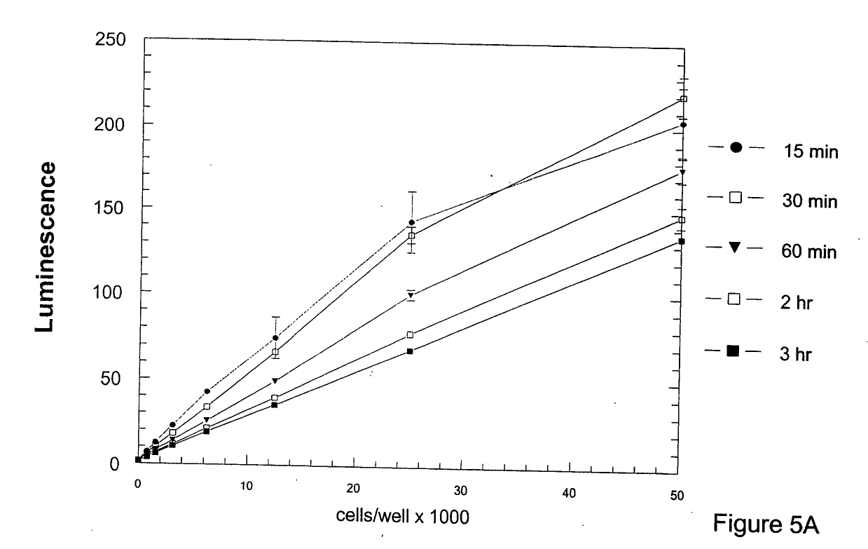

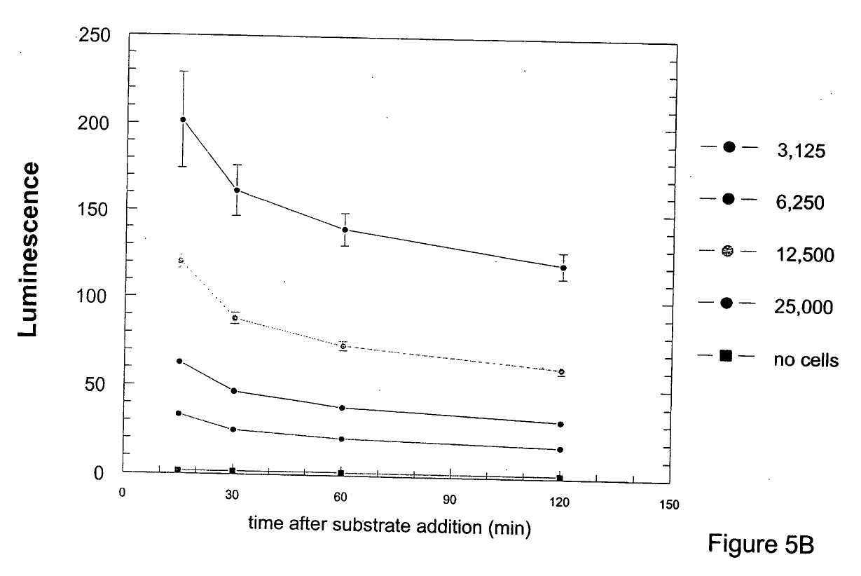

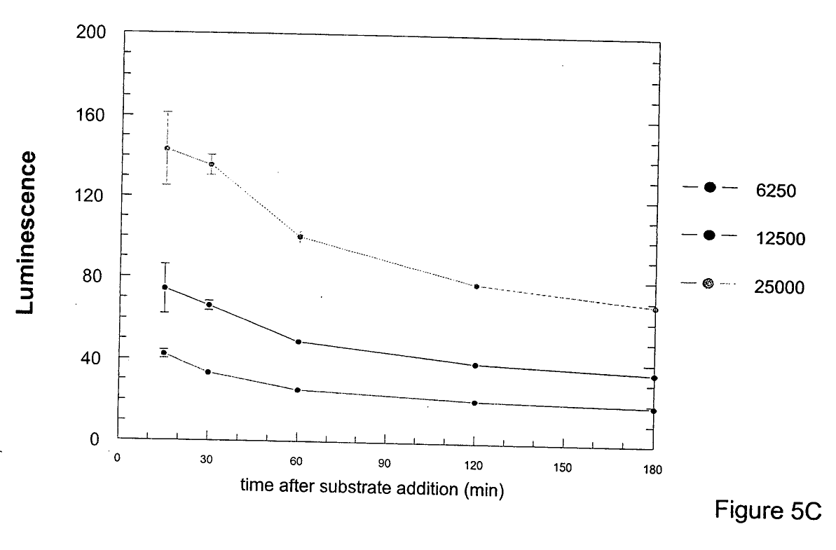

The linearity and kinetics of the luminescent assay for LLVY (SEQ ID NO:1) activity was determined (FIGS. 5A-C). Although under the tested conditions the assay was not linear over all time points with respect to cell number, proteasome assay conditions may be improved by increasing the Mg and/or substrate concentrations in the assay mixture so as to stabilize the proteasome, increase activity and/or provide a longer luminescence or “glow” to the assay.

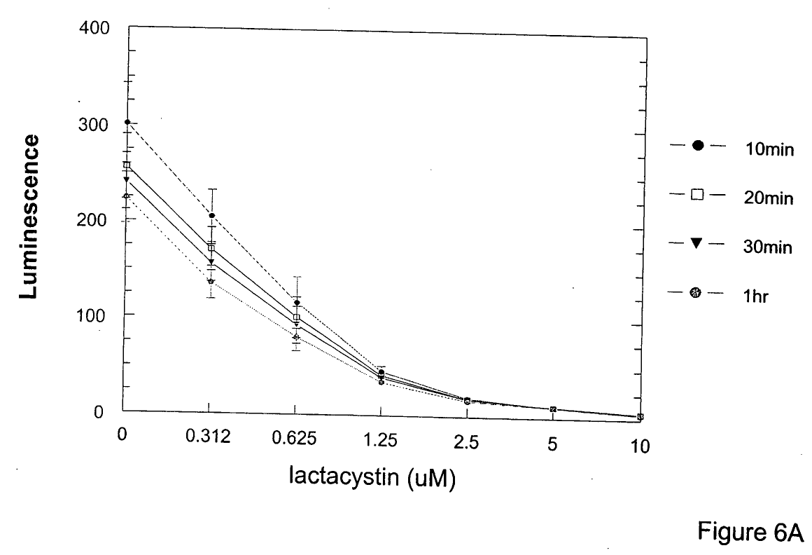

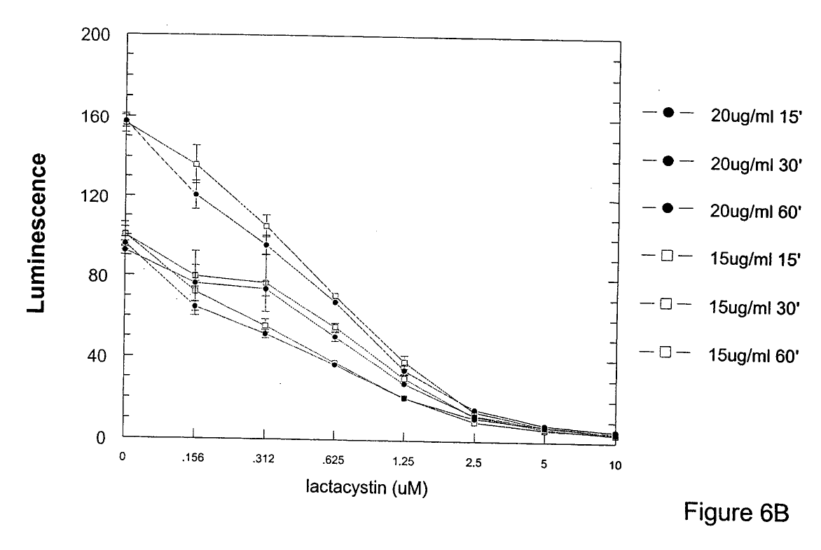

FIGS. 6A-B show the inhibition of luminescence over time by lactacystin in U937 (A) and HL-60 (B) cells. Similar IC50 values were obtained over a wide window.

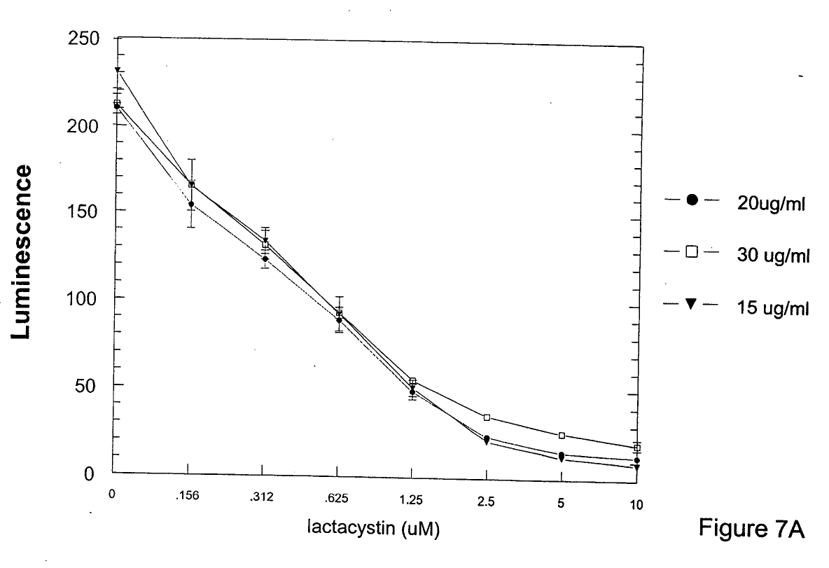

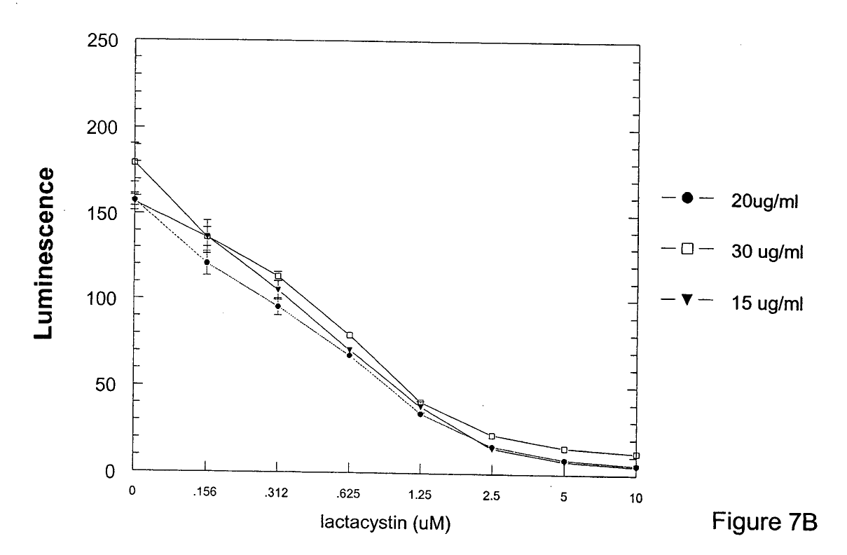

FIGS. 7A-B show multiple reads of the same plate treated with lactacystin in medium containing 10% fetal bovine serum (FBS) and 15 to 30 μg/ml digitonin. Digitonin permeabilized cells at various low concentrations when the cells were in either 5% or 10% fetal bovine serum, and nearly all of the protease activity continued to be inhibitable with lactacytsin.

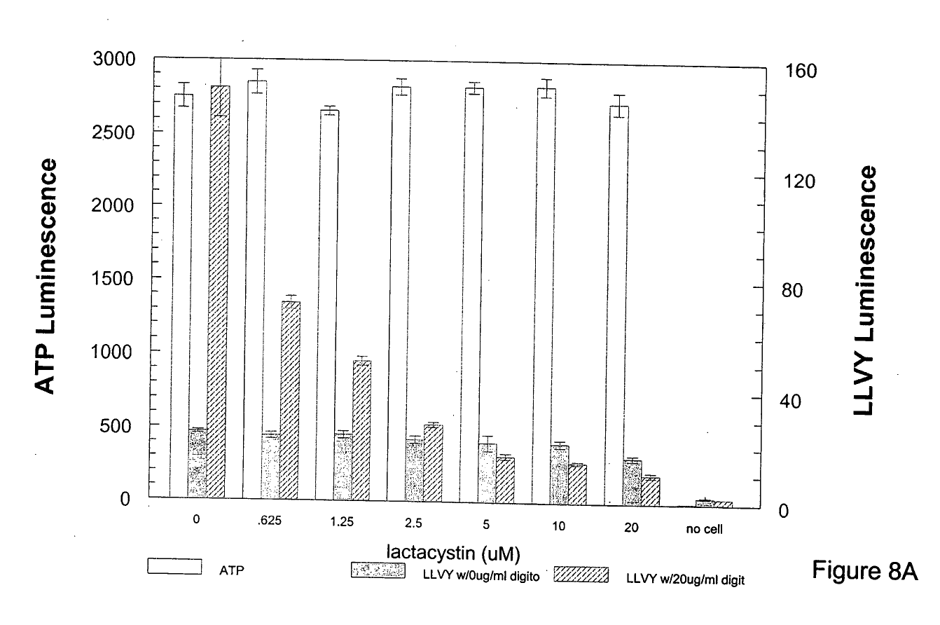

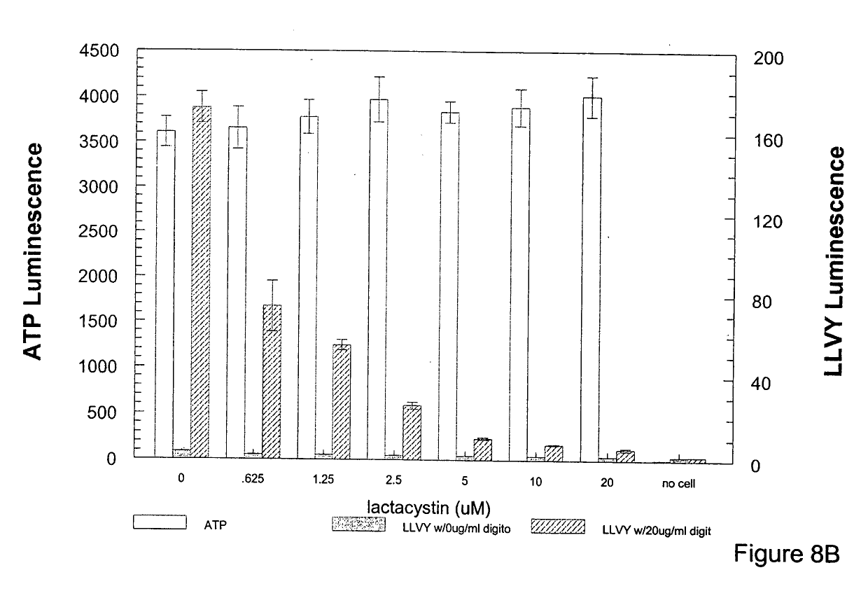

A comparison of proteasome chymotrypsin activity in HL-60 cells or U937 cells treated with various concentrations of lactacystin in the presence or absence of digitonin (0 and 20 μg/ml digitonin) is shown in FIGS. 8A-B. At the concentration of lactacystin tested, drug treatment did not kill the cells (measured by an ATP assay, CellTiterGlo®, Promega Corp., Madison, Wis.).

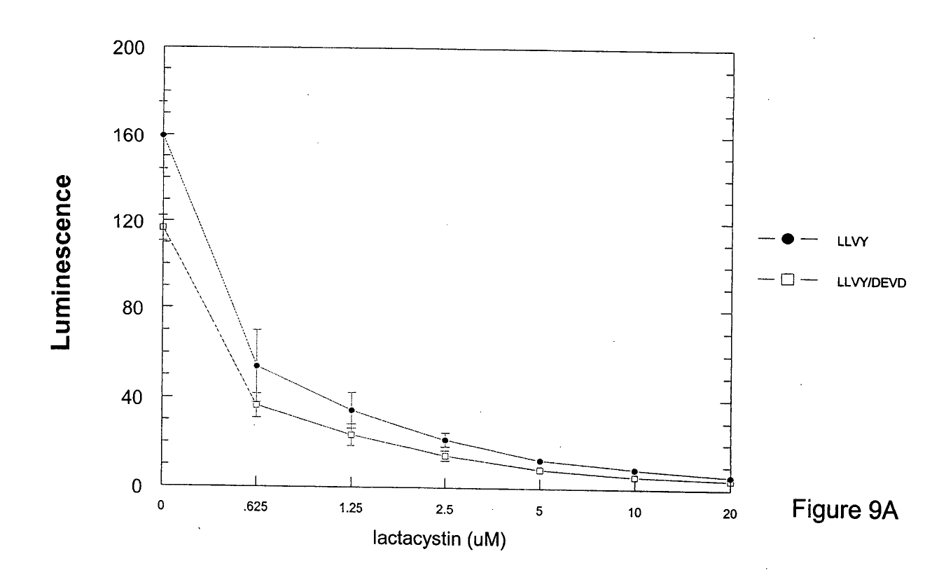

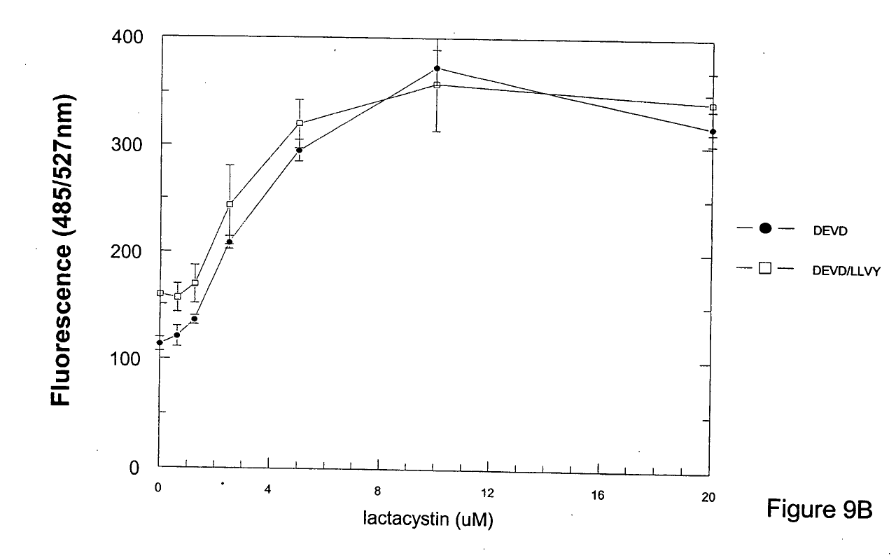

Results for a multiplex assay using a luminogenic proteasome substrate and a fluorogenic (R110) caspase-3 substrate are shown in FIG. 9. Cells were treated for a longer duration with lactacystin to induce caspase activity. The graph shows that similar proteasome inhibition curves were achieved with and without the fluorogenic caspase substrate present in the reagent containing the luminogenic proteasome substrate indicating that both proteasome activity and apoptotic activity may be measured.

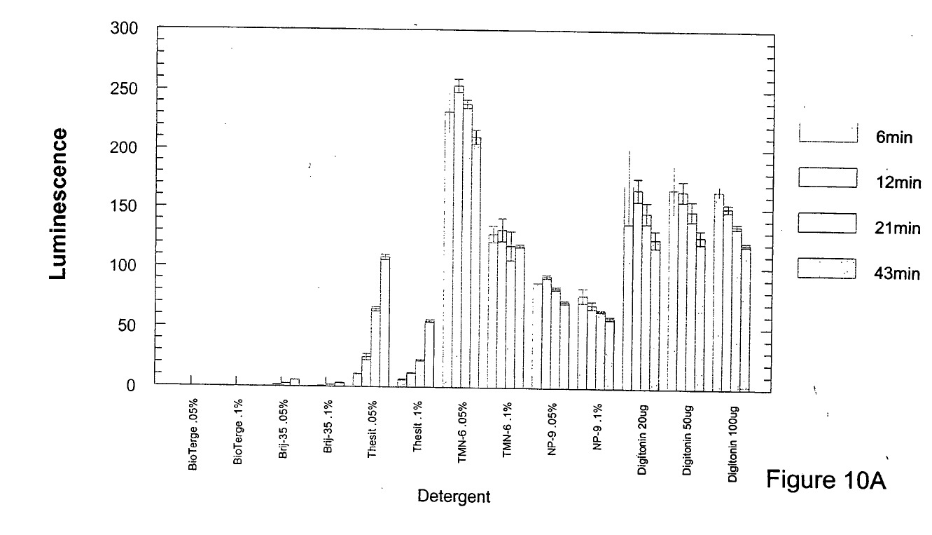

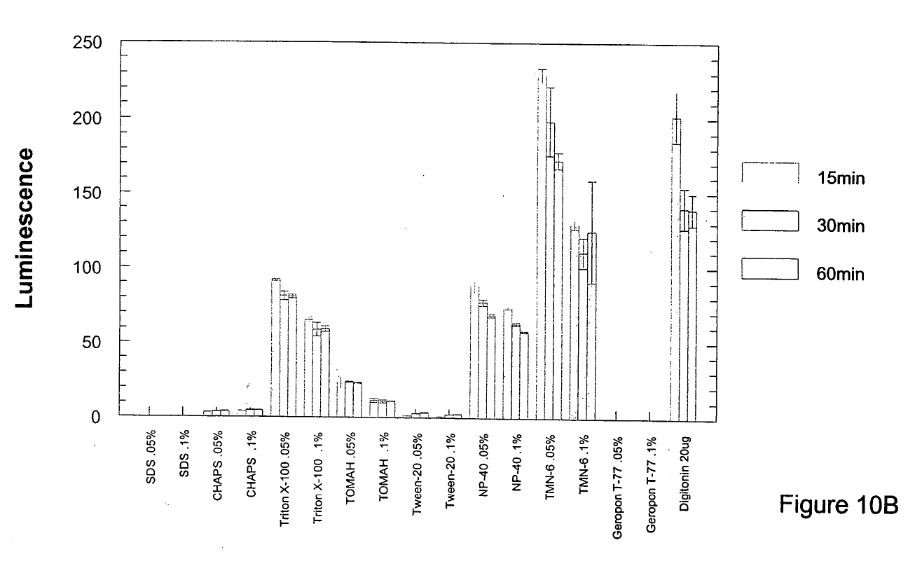

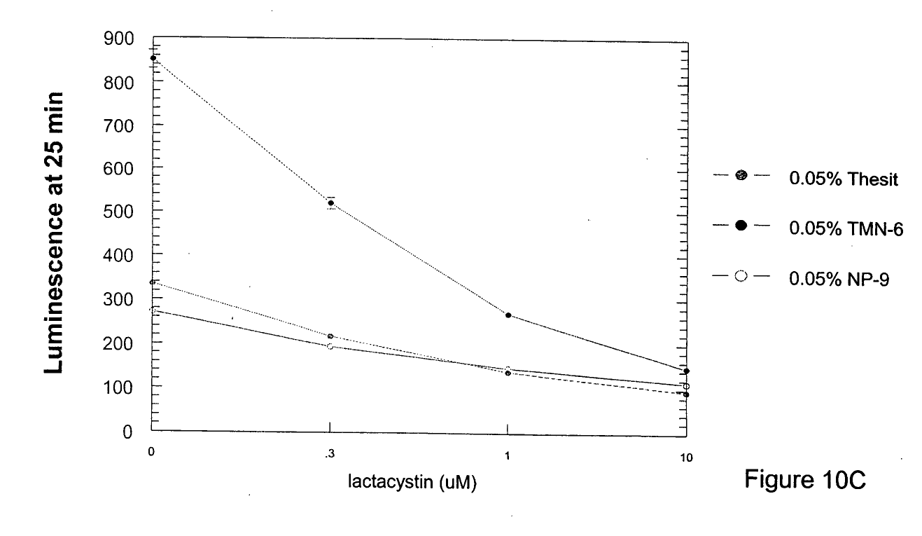

Other permeabilizing agents were screened for those suitable to detect proteasome activity in a cell-based assay (FIGS. 10A-C). Reagents containing low concentrations of various detergents (0.05 and 0.1% final) were prepared. 100 μl/well samples containing 25,000 HL-60 cells in complete medium were added to a 96-well plate and equilibrated at 37° C. The plate and reagents were cooled to 22° C. and an equal volume of reagent was added to each sample. The plate was shaken for 1 minute, then incubated at 22° C. Luminescence was then determined over time. SDS, CHAPS, TOMAH®, Tween®-20, Geropon® T-77, BioTerge and Brij-35 had negative effects on either the proteasome or the luciferase. Several detergents, including Thesit®, Tergitol® TMN-6, Tergitol® NP-9, Triton X-100 and NonIdet-40 did not substantially effect proteasome or luciferase activity, although this assay does not indicate the source of the protease(s) cleaving the LLVY-aminoluciferin (SEQ ID NO:1) substrate.

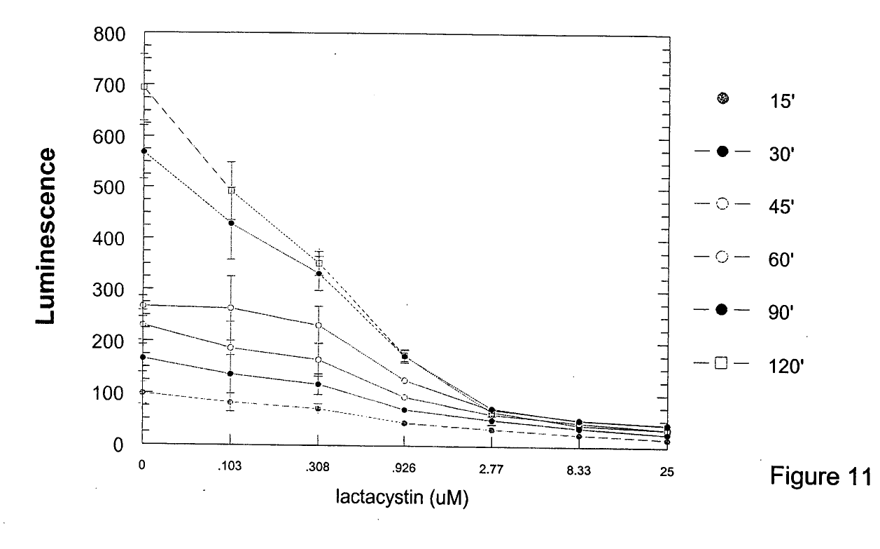

FIG. 11 shows luminescence from H226 cells treated with lactacystin, LLVY-aminoluciferin (SEQ ID NO:1) and 0.04% TMN-6. TMN-6 at 0.05% was comparable to 20 μg/ml digitonin in U937 cells but was not sufficient for a proteasome assay when present at a concentration of 0.04% with H226 cells. U937 cells were also treated with 100 μg/ml saponin, however, at later time points the luminescent signal was not linear.

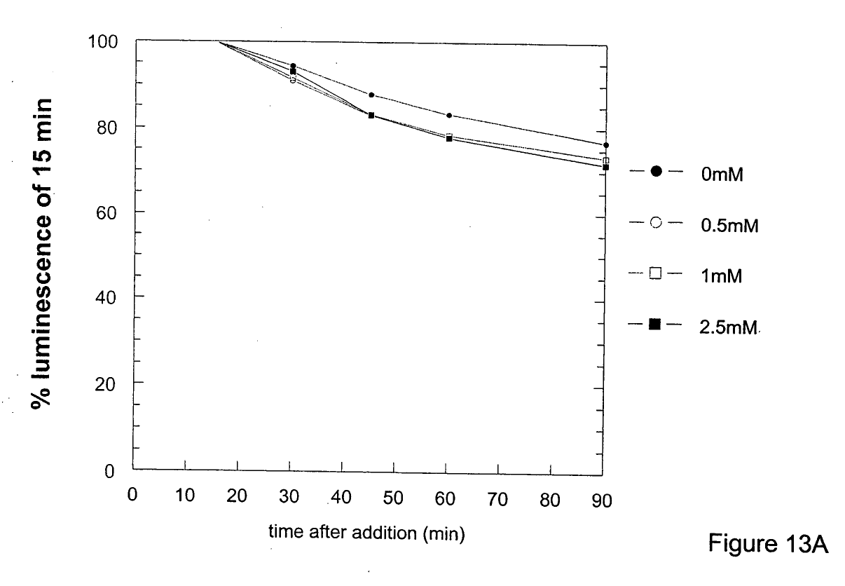

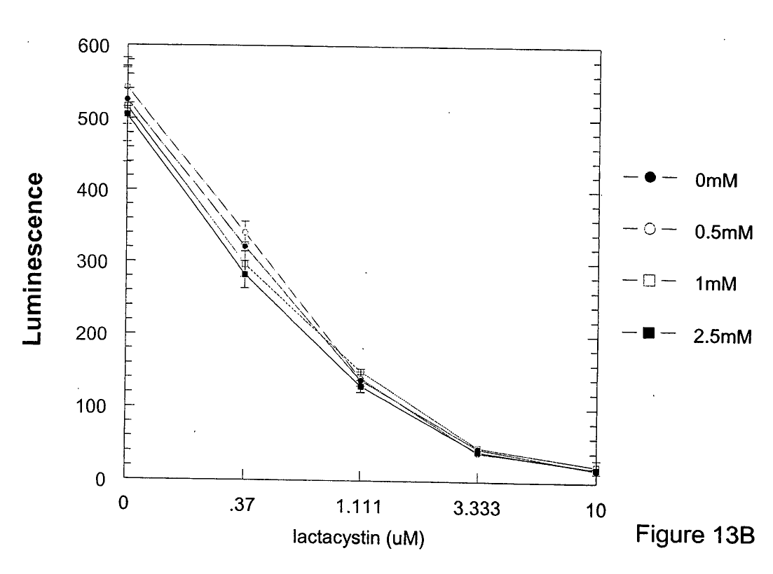

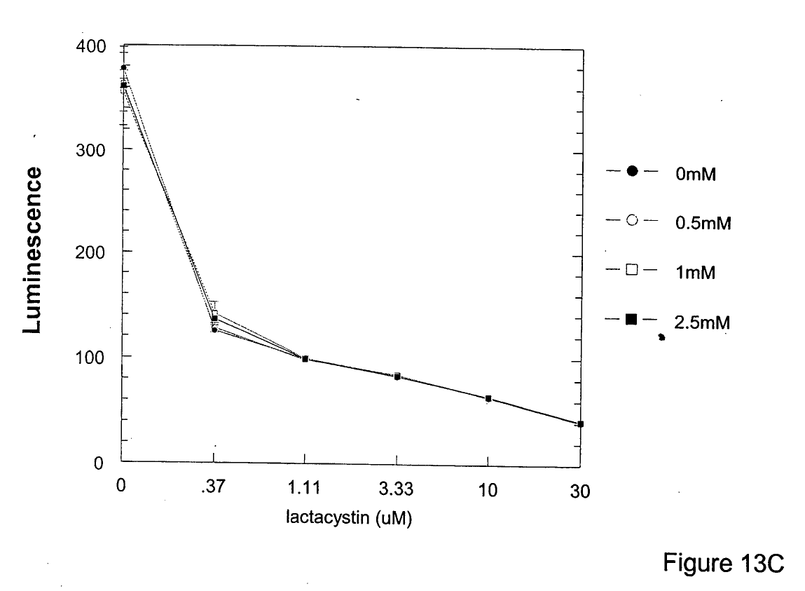

Mg, EDTA concentrations, and pH were varied to optimize reaction conditions. In particular, Mg concentrations appear to influence chymotrypsin activity, and EDTA concentrations appear to alter numerous proteolytic activities. Data on the effect of Mg concentration on luminescent or fluorescent assays, or EDTA on luminescent assays, to detect proteasomes is shown in FIGS. 12 and 13.

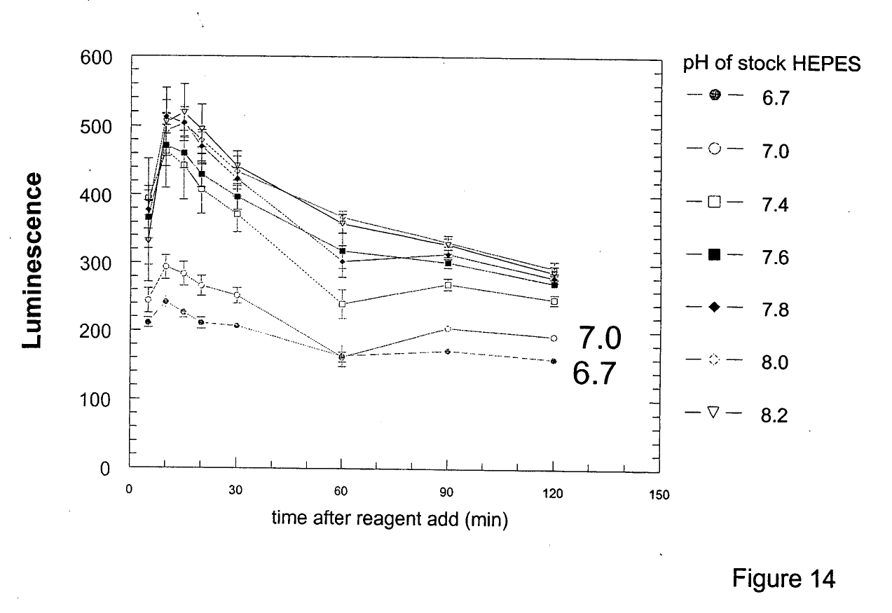

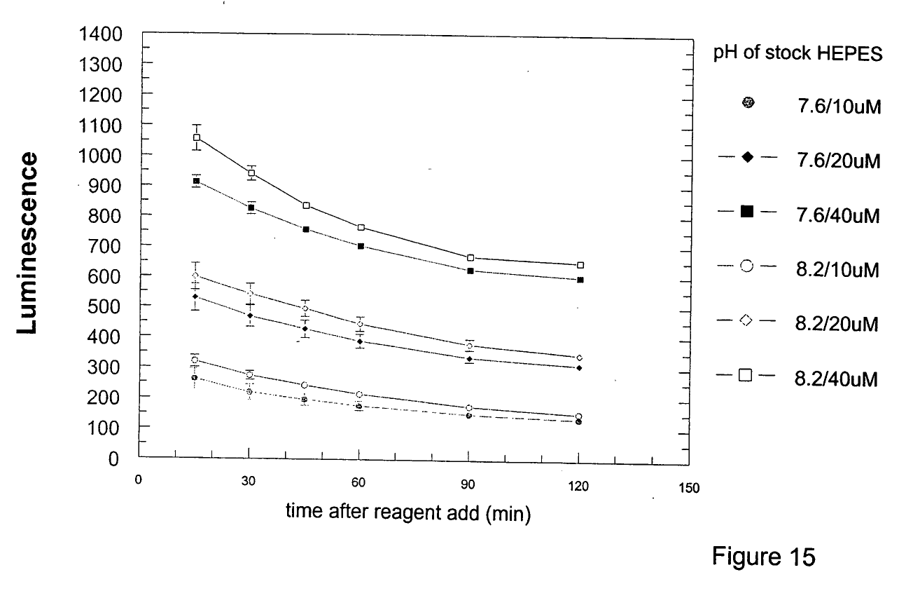

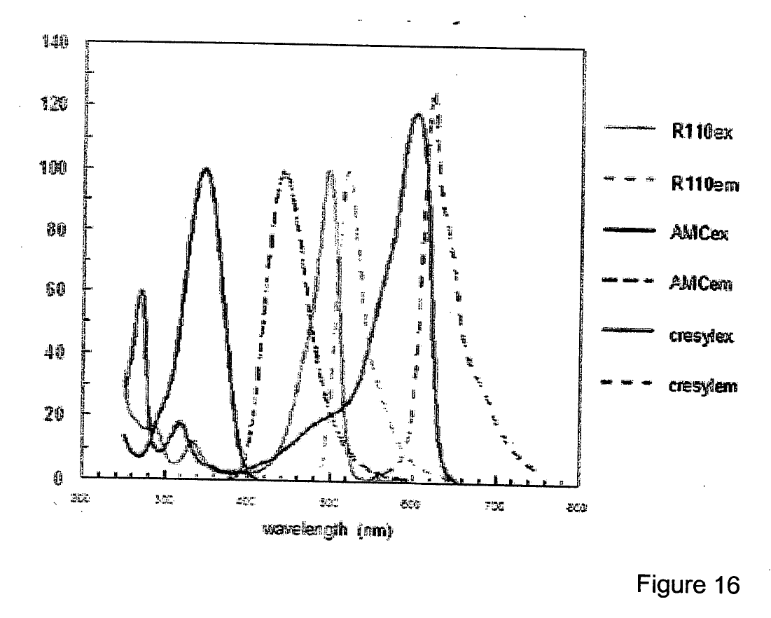

The results for varying pH and substrate concentration are shown in FIGS. 14-15. Based on the data, greater substrate concentration apparently improved half-life.

### REFERENCES

Bond et al., Ann. Rev. Biochem., 56:333 (1987).

Constam et al., J. Biol. Chem., 270:26931 (1995).

Cook et al., Anal. Biochem., 179:1 (1989).

Fenteany et al., Science, 268:726 (1995).

Fernandes-Alnemri et al., PNAS USA, 93:7464 (1996).

Haunstetter et al., Circ. Res., 82:1111 (1998).

Masuda-Nishimura et al., Lett. Appl. Microbio., 30:130 (2000).

Mellgren, J. Biol. Chem., 272:29899 (1997).

Miska and Geiger, J. Clin. Chem. Clin. Biochem., 25:23 (1989).

Monsees et al., Anal. Biochem., 221:329 (1994).

Monsees et al., J. Biolum. Chemilum., 10:213 (1995).

Myers, J. Immunol. Methods, 212: 99 (1998).

Nicholson et al., Nature, 376:37 (1995).

Ramsby et al., (1994) Electrophoresis, 15:265 (1994).

Riss et al., Assay and Drug Development Technologies, 2:1 (2004).

Syntichaki et al., Nature Reviews, 4:672 (2003).

Tewari et al., Cell, 81:801 (1995).

Thornberry et al., Nature, 356:768 (1992).

Tran et al., Archives of Biochemistry and Biophysics, 403:160 (2002).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed:

1. A method to detect one or more proteasome-specific proteolytic activities associated with proteasomes, comprising:
a) providing a reaction mixture for a beetle luciferase-mediated reaction comprising eukaryotic cells, a cell membrane permeabilization reagent in an amount which does not substantially disrupt intracellular membrane bound organelles or compartments in the cells, and a luminogenic substrate for a proteasome associated protease, wherein the proteolysis of the luminogenic substrate by the protease yields a substrate for the beetle luciferase; and
b) detecting luminescence in the reaction mixture.

2. A method to detect one or more proteasesome-specific proteolytic activities associated with proteasomes, comprising:
a) contacting a sample comprising intact eukaryotic cells with a reaction mixture for a beetle luciferase-mediated reaction which comprises a luminogenic substrate for a proteasome associated protease and a cell membrane permeabilization reagent in an amount which does not substantially disrupt intracellular membrane bound organelles or compartments in the cells, so as to yield a mixture, wherein the proteolysis of the luminogenic substrate by the protease yields a substrate for the beetle luciferase; and
b) detecting luminescence in the mixture.

3. The method of claim 1 or 2 wherein the luminogenic substrate is a chymotrypsin substrate.

4. The method of claim 1 or 2 wherein the luminogenic substrate is a trypsin substrate.

5. The method of claim 1 or 2 wherein the luminogenic substrate is a caspase substrate.

6. The method of claim 1 or 2 wherein the luminogenic substrate comprises LLVY (SEQ ID NO:1).

7. The method of claim 1 or 2 wherein the luminogenic substrate comprises LRR.

8. The method of claim 1 or 2 wherein the luminogenic substrate comprises nLPnLD (SEQ ID NO:2).

9. The method of claim 1 or 2 further comprising contacting the mixture with a second reaction mixture for a second enzyme-mediated reaction which comprises a fluorogenic substrate for the second enzyme.

10. The method of claim 9 further comprising detecting fluorescence.

11. The method of claim 10 wherein fluorescence is employed to detect the presence or amount of a co-factor, substrate or enzyme for the second enzyme-mediated reaction.

12. The method of claim 10 wherein luminescence and fluorescence are detected sequentially.

13. The method of claim 1 or 2 wherein the reaction mixture further comprises a fluorogenic substrate for a second enzyme-mediated reaction.

14. The method of claim 13 further comprising detecting fluorescence.

15. The method of claim 14 wherein fluorescence is employed to detect the presence or amount of a co-factor, substrate or enzyme for the second enzyme-mediated reaction.

16. The method of claim 10 wherein luminescence and fluorescence are detected concurrently.

17. The method of claim 14 wherein luminescence and fluorescence are detected concurrently.

18. The method of claim 14 wherein luminescence and fluorescence are detected sequentially.

19. The method of claim 1 or 2 wherein the cells are lysed after luminescence is detected.

20. The method of claim 9 or 13 wherein the fluorogenic substrate comprises ethidium bromide, fluorescein, Cy3, BODIPY, a rhodol, Rox, 5-carboxyfluorescein, 6-carboxyfluorescein, an anthracene, 2-amino-4-methoxynapthalene, a phenalenone, an acridone, fluorinated xanthene derivatives, α-naphtol, β-napthol, 1-hydroxypyrene, coumarin, 7-amino-4-methylcoumarin (AMC), 7-amino-4-trifluoromethylcoumarin (AFC), Texas Red, tetramethylrhodamine, carboxyrhodamine, rhodamine, cresyl violet, rhodamine-110 or resorufin.

21. The method of claim 9 or 13 wherein the second enzyme-mediated reaction is mediated by a glycosidase, phosphatase, kinase, dehydrogenase, peroxidase, sulfatase, peptidase, transferase, hydroxylase, dealkylase, dehalogenase, deamidase, or hydrolase.

22. The method of claim 9 or 13 wherein the second enzyme-mediated reaction is mediated by a protease.

23. The method of claim 22 wherein the second enzyme is a caspase.

24. The method of claim 23 wherein the caspase includes caspase-3 or caspase-7.

25. The method of claim 1 or 2 further comprising contacting the mixture with a composition comprising a reagent to detect a cellular molecule.

26. The method of claim 25 wherein the reagent that detects a cellular molecule detects nucleic acid or protein.

27. The method of claim 25 further comprising detecting the cellular molecule.

28. The method of claim 27 wherein fluorescence is employed to detect the cellular molecule.

29. The method of claim 25 wherein the reagent is fluorogenic.

30. The method of claim 25 wherein the reagent comprises ethidium bromide, propidium iodide, or acridine orange.

31. The method of claim 25 wherein the reagent comprises a nucleic acid binding dye.

32. The method of claim 1 or 2 further comprising contacting the mixture with a second reaction mixture to detect a moiety associated with a nonenzymatic reaction.

33. The method of claim 32 wherein the nonenzymatic reaction includes binding of the moiety to another molecule.

34. A method to detect two or more cytosolic activities in a cell, comprising:
a) providing a reaction mixture comprising eukaryotic cells, a fluorogenic or a luminogenic substrate for a proteasome associated protease, a second substrate for a cytosolic enzyme that is not associated with proteasomes, and a cell membrane permeabilization reagent in an amount which does not substantially disrupt intracellular membrane bound organelles or compartments in the cells; and
b) detecting in the reaction mixture luminescence or fluorescence and the presence or amount of the cytosolic enzyme that is not associated with proteasomes, wherein luminescence or fluorescence correlates with the activity of the proteasome associated protease.

35. A method to detect two or more cytosolic activities in a cell, comprising:
a) contacting a sample comprising intact eukaryotic cells with a reaction mixture comprising a fluorogenic or a luminogenic substrate for a proteasome specific protease, a substrate for a cytosolic enzyme that is not associated with proteasomes, and a cell membrane permeabilization reagent in an amount which does not substantially disrupt intracellular membrane bound organelles or compartments in the cells, so as to yield a mixture; and
b) detecting in the mixture luminescence or fluorescence and the presence or amount of the cytosolic enzyme that is not associated with proteasomes, wherein luminescence or fluorescence correlates with the activity of the proteasome associated protease.

36. The method of claim 34 or 35 wherein the fluorogenic or luminogenic substrate is a chymotrypsin substrate.

37. The method of claim 34 or 35 wherein the fluorogenic or luminogenic substrate is a trypsin substrate.

38. The method of claim 34 or 35 wherein the fluorogenic or luminogenic substrate is a caspase substrate.

39. The method of claim 34 or 35 wherein the fluorogenic or luminogenic substrate comprises LLVY (SEQ ID NO:1).

40. The method of claim 34 or 35 wherein the fluorogenic or luminogenic substrate comprises LRR.

41. The method of claim 34 or 35 wherein the fluorogenic or luminogenic substrate comprises nLPnLD (SEQ ID NO:2).

42. The method of claim 34 or 35 wherein the substrate for the protease associated with proteasomes is a luminogenic substrate which, after proteolysis by the protease, yields a substrate for a beetle luciferase and the second substrate is a fluorogenic substrate.

43. The method of claim 42 wherein luminescence and fluorescence are detected sequentially.

44. The method of claim 42 wherein luminescence and fluorescence are detected concurrently.

45. The method of claim 34 or 35 wherein the substrate for the protease associated with proteasomes is a fluorogenic substrate and the second substrate which, after proteolysis by the protease, yields a substrate is a luminogenic substrate for a beetle luciferase.

46. The method of claim 45 wherein luminescence and fluorescence are detected sequentially.

47. The method of claim 45 wherein luminescence and fluorescence are detected concurrently.

48. The method of claim 45 wherein the cells are lysed after fluorescence is detected.

49. The method of claim 42 wherein the cells are lysed after luminescence is detected.

50. The method of claim 41 wherein the fluorogenic substrate comprises ethidium bromide, fluorescein, Cy3, BODIPY, a rhodol, Rox, 5-carboxyfluorescein, 6-carboxyfluorescein, an anthracene, 2-amino-4-methoxynapthalene, a phenalenone, an acridone, fluorinated xanthene derivatives, α-naphtol, β-napthol, 1-hydroxypyrene, coumarin, 7-amino-4-methylcoumarin (AMC), 7-amino-4-trifluoromethylcoumarin (AFC), Texas Red, tetramethylrhodamine, carboxyrhodamine, rhodamine, cresyl violet, rhodamine-110 or resorufin.

51. The method of claim 45 wherein the fluorogenic substrate comprises ethidium bromide, fluorescein, Cy3, BODIPY, a rhodol, Rox, 5-carboxyfluorescein, 6-carboxyfluorescein, an anthracene, 2-amino-4-methoxynapthalene, a phenalenone, an acridone, fluorinated xanthene derivatives, α-naphtol, β-napthol, 1-hydroxypyrene, coumarin, 7-amino-4-methylcoumarin (AMC), 7-amino-4-trifluoromethylcoumarin (AFC), Texas Red, tetramethylrhodamine, carboxyrhodamine, rhodamine, cresyl violet, rhodamine-110 or resorufin.

52. The method of claim 34 or 35 wherein the cytosolic enzyme is a glycosidase, phosphatase, kinase, dehydrogenase, peroxidase, sulfatase, peptidase, transferase, hydroxylase, dealkylase, dehalogenase, deamidase, or hydrolase.

53. The method of claim 34 or 35 wherein a reaction mixture comprising a luminogenic substrate is a reaction mixture for a beetle luciferase-mediated reaction.

54. The method of claim 1, 2, 34 or 35 wherein the cell membrane permeabilizing reagent is digitonin.

55. The method of claim 54 wherein digitonin is present at about 10 μg/ml to 40 μg/ml.

56. A method to identify a modulator of a proteasome-specific proteolytic activity, comprising
a) contacting one or more agents, eukaryotic cells, and a reaction mixture for a beetle luciferase-mediated reaction comprising a cell membrane permeabilization reagent in an amount which does not substantially disrupt intracellular membrane bound organelles or compartments in the cells, and a luminogenic substrate for a proteasome associated protease, so as to yield a mixture, wherein the proteolysis of the luminogenic substrate by the protease yields a substrate for the beetle luciferase; and
b) comparing luminescence in the mixture to luminescence in a corresponding mixture which lacks the one or more agents.

57. The method of claim 56 wherein the one or more agents inhibit the activity of proteasomes.

58. A kit comprising:
a buffer comprising a cell membrane permeabilization reagent which, in an effective amount in a reaction mixture comprising eukaryotic cells, does not substantially disrupt intracellular membrane bound organelles or compartments in the cells; and
a luminogenic or fluorogenic substrate for a proteasome associated protease.

59. The kit of claim 58 further comprising a substrate for an enzyme that is not associated with proteasome.

60. The kit of claim 58 wherein the luminogenic substrate, once cleaved by the protease associated with proteasomes, yields a substrate for a beetle luciferase.
